(19) United States
(12) Patent Application Publication
(10) Pub. No.: US 2022/0225894 A1
(43) Pub. Date: Jul. 21, 2022

(54) WEARABLE RESISTANCE DEVICE WITH POWER MONITORING

(71) Applicant: Tau Orthopedics, Inc., Rancho Santa Fe, CA (US)

(72) Inventors: Jacob A. Moebius, Solana Beach, CA (US); Belinko K. Matsuura, Solana Beach, CA (US); David G. Matsuura, Solana Beach, CA (US); Gerard von Hoffmann, Rancho Santa Fe, CA (US)

(21) Appl. No.: 17/494,250

(22) Filed: Oct. 5, 2021

Related U.S. Application Data
(63) Continuation of application No. 15/593,138, filed on May 11, 2017, now abandoned.
Application No. 15/593,138 is a continuation-in-part of application No. 15/078,250, filed on Mar. 23, 2016, now Pat. No. 9,656,117.

(51) Int. Cl. A61B 5/11; A63B 21/00; A63B 71/06; A63B 23/04; A41D 31/00; A41D 1/00; A63B 21/008

(52) U.S. Cl. CPC A61B 5/112; A41D 27/205; A63B 21/4025; A63B 71/0619; A63B 23/0405; A41D 31/00; A41D 1/002; A63B 21/00845; A63B 21/4039; A63B 23/0482; A63B 23/0494; A63B 21/4017; A63B 2220/34; A63B 2225/20; A63B 2220/62; A63B 2220/22; A63B 2220/54; A63B 2220/836; A63B 21/4011

(57) ABSTRACT

Disclosed is a technical training garment configured for use with modular, interchangeable biomechanics units and or resistance modules. The garment may provide resistance to movement throughout an angular range of motion and or tracks a variety of biomechanical parameters such as stride length, stride rate, angular velocity and power expended by the wearer. The garment may be low profile, and worn by a wearer as a primary garment or beneath or over conventional clothing or athletic uniform. The device may be worn as a supplemental training and or diagnostic tool during conventional training protocols, or as a biomechanics or biometric data capture device during competition.

### CROSS-REFERENCE TO RELATED APPLICATIONS

This application is a continuation of U.S. patent application Ser. No. 15/593,138, filed May 11, 2017, which is a continuation-in-part of U.S. patent application Ser. No. 15/078,250, filed Mar. 23, 2016, the entirety of which is hereby expressly incorporated by reference herein.

### BACKGROUND OF THE INVENTION

Resistance training, sometimes known as weight training or strength training, is a specialized method of conditioning designed to increase muscle strength, muscle endurance, tone and muscle power. Resistance training refers to the use of any one or a combination of training methods which may include resistance machines, dumbbells, barbells, body weight, and rubber tubing.

The goal of resistance training, according to the American Sports Medicine Institute (ASMI), is to “gradually and progressively overload the musculoskeletal system so it gets stronger.” This is accomplished by exerting effort against a specific opposing force such as that generated by elastic resistance (i.e. resistance to being stretched or bent). Exercises are isotonic if a body part is moving against the force. Exercises are isometric if a body part is holding still against the force. Resistance exercise is used to develop the strength and size of skeletal muscles. Full range of motion is important in resistance training because muscle overload occurs only at the specific joint angles where the muscle is worked. Properly performed, resistance training can provide significant functional benefits and improvement in overall health and well-being.

Research shows that regular resistance training will strengthen and tone muscles and increase bone mass. Resistance training should not be confused with weightlifting, power lifting or bodybuilding, which are competitive sports involving different types of strength training with non-elastic forces such as gravity (weight training or plyometrics) an immovable resistance (isometrics, usually the body's own muscles or a structural feature such as a door frame).

Whether or not increased strength is an objective, repetitive resistance training can also be utilized to elevate aerobic metabolism, for the purpose of weight loss, and to enhance muscle tone.

Resistance exercise equipment has therefore developed into a popular tool used for conditioning, strength training, muscle building, and weight loss. Various types of resistance exercise equipment are known, such as free weights, exercise machines, and resistance exercise bands or tubing.

Various limitations exist with the prior art exercise devices. For example, many types of exercise equipment, such as free weights and most exercise machines, are not portable. With respect to exercise bands and tubing, they may need to be attached to a stationary object, such as a closed door or a heavy piece of furniture, and require sufficient space. This becomes a problem when, for example, the user wishes to perform resistance exercises in a location where such stationary objects or sufficient space are not readily found.

Resistance bands are also limited to a single resistance profile in which the amount of resistance changes as a function of angular displacement of the joint under load. This may result in under working the muscles at the front end of a motion cycle, and over working the muscles at the back end of the cycle. Conventional elastic devices also provide a unidirectional bias that varies in intensity throughout an angular range but not in direction. Such devices thus cannot work both the flexor and extensor muscles of a given motion segment without adjustment, and may be uncomfortable due to the constant bias even in the absence of motion.

A need therefore exists for low profile resistance based wearable toning garments that may be used on their own without the need to employ other types of equipment, that free the wearer for other simultaneous activities, and that can apply a non-elastic load throughout both a flexion and extension range of motion.

A need also exists for wearable devices that can determine various biometrics with or without imposition of resistance to movement.

### SUMMARY OF THE INVENTION

There is provided in accordance with one aspect of the present invention a wearable garment training system for monitoring stride biomechanics at a motion segment or joint such as the hip. The system comprises a waist portion; a left leg portion; and a right leg portion. A left hip biomechanics unit may be removably carried by a left connector on the garment and aligned with a rotational axis of the left leg, and a right hip biomechanics unit may be removably carried by a right connector on the garment and aligned with a rotational axis of the right leg. The left and right biomechanics units each capture data for enabling the determination of at least one biomechanics metric of motion such as power expended by the wearer throughout a range of motion at the hip.

The system is configured to capture any of the raw biometric data discussed elsewhere herein. The raw data may be partially or fully processed in a processor within the biomechanics unit or electronics module on the garment, or raw, partially or fully processed data may be exported to an external, remote processor for determination (computation) of derived values for display to a wearer or coach and or storage in memory for comparison or progress tracking purposes. The remote processor may be cloud based, or carried within a smart phone or computer.

At least one of the biomechanics units may be configured to capture time and angle data during flexion and or extension, and or may be configured to measure force applied during flexion and or extension. At least one of the biomechanics units may be configured to capture data relating to angular velocity of a wearer's leg throughout the range of motion. Preferably all data capture will be bilaterally symmetrical, although 180 degrees out of phase due to normal stride mechanics.

A processor may be provided, for determining a metric such as power expended throughout the range of motion. The system may additionally comprise a transmitter, for transmitting processed or preprocessed (raw) data such as time and angle data to a remote device. The system may further comprise a left knee biomechanics unit and a right knee biomechanics unit. The left and right hip biomechanics units may each further comprise rotatable resistance units such as rotary viscous dampers. The left and right resistance units may each impose a resistance of at least about 5 inch pounds, at least about 10 inch pounds, or at least about 15 inch pounds.

The garment may comprise a compression fabric, and may comprise a polyester elastane fabric with moisture wicking properties. The garment may comprise a wearable harness. The harness may comprise a waist band and left and right leg bands. Each biomechanics unit may comprise a housing and a femoral lever extending from the housing.

The system may additionally comprise a short range WiFi or Bluetooth transmitter or a cellular transmitter. In one implementation the system comprises an ANT+ transmitter. Each biomechanics unit may be configured to capture data for enabling the determination of stride length, and or stride rate and or actual distance travelled computed based upon stride count and stride length. At least one biomechanics unit comprises a strain gauge and or a torque sensor. The left and right biomechanics units may be configured to capture data reflecting left side and right side asymmetries in performance, such as left side and right side asymmetries in power output and or left side and right side asymmetries in stride length.

The system may additionally comprise a processor configured to determine power to heart rate ratio, or configured to transmit data enabling the determination of power to heart rate ratio such as to move the computation off board to a remote processor. The system may further comprise a processor configured to determine power to weight ratio, or to transmit data enabling the determination of power to weight ratio on a remote processor. The processor may be configured to determine efficiency factor, or to transmit data enabling the determination of efficiency factor.

The system may further comprise an electronics module carried by the garment, attached to or integrated into a biomechanics unit or carried separately by the garment. The electronics module may comprise a processor, a transmitter, a power supply and other electronics disclosed herein. The electronics module may be in wired communication with at least one biomechanics unit, and may be permanently or removably carried by the waist portion.

There is provided in accordance with another aspect of the present invention, a wearable garment training system for monitoring power expended by a wearer. The wearable garment training system comprising a waist portion, a left leg portion, and a right leg portion. A left hip biomechanics unit may be carried by a left connector on the garment and aligned with a rotational axis of the left leg, and a right hip biomechanics unit may be carried by a right connector on the garment and aligned with a rotational axis of the right leg.

One or more hardware processors may be provided, wherein the one or more hardware processors are configured to receive a first measurement of time and angular rotation data from a left biomechanics unit, and receive a second measurement of time and angular rotation data from a right biomechanics unit. The processor is configured to apply power processing rules on the first measurement of time and angular rotation data and the second measurement of time and angular rotation data, and determine power generated by the wearer based on the application of the power processing rules on the first measurement of time and angular rotation data and the second measurement of time and angular rotation data. The one or more hardware processors may be further configured to generate a display including an indication of the determined power and or other metrics disclosed herein and transmit the generated display to a computing device.

Further features and advantages of the present invention will become apparent to those of skill in the art in view of the detailed description of preferred embodiments which follows, when considered together with attached drawings and claims.

### BRIEF DESCRIPTION OF THE DRAWINGS

FIG. 1 is a side elevational view of a toning and/or power measurement garment showing a right hip and a right knee resistance unit.

FIG. 2 is a plan view of a toning garment resistance unit.

FIG. 3 is a side elevational view of the resistance unit of FIG. 2.

FIG. 4 is a side elevational view of an alternate configuration of the resistance unit of FIG. 2.

FIG. 5 is a resistance unit as in FIG. 2, attached to a garment with force distribution layers.

FIG. 6 is a side elevational view of the resistance unit and garment assembly of FIG. 5.

FIG. 7 is a side elevational view of an alternate configuration of the resistance unit and garment assembly of FIG. 5.

FIG. 8 is a resistance unit secured to a garment, showing an alternative reinforced femoral attachment configuration.

FIG. 9 is a side elevational view of a resistance unit having a superior connector, an inferior, femoral connector and a resistance element.

FIG. 10 is an exploded view of the resistance unit of FIG. 9.

FIG. 11 is a side elevational view of a left side resistance unit, having a posterior connector for connection to a right side resistance unit.

FIG. 12 is a perspective view of a detachable, modular resistance unit, having a resistance element and a femoral lever arm.

FIG. 13 is a side elevational view of a lower body garment, having a resistance unit docking station aligned with the hip.

FIG. 14 is a detail view taken along the line 14-14 in FIG. 13.

FIG. 15 is a garment as in FIG. 13, with a removable modular resistance unit partially assembled with the garment.

FIG. 16 is a garment as in FIG. 15, with the removable modular resistance unit fully installed, and engaged with the docking station.

FIG. 17 is a side view of an athletic training garment incorporating hip and knee resistance units and technical fabric features of the present invention.

FIG. 18 is an exploded perspective view of a first lever having a resistance unit thereon, and a docking platform having a second lever.

FIG. 19 is a perspective view of a docking platform having a second lever, attached to a force transfer layer.

FIG. 20 is a perspective view of a resistance subassembly, including an upper lever attached to a force transfer layer, and a lower lever having a resistance unit pivotably mounted on the docking station.

FIG. 21 is a side elevational view of first and second levers configured to receive a resistance unit having a compound post thereon.

FIG. 22 is a side elevational view as in FIG. 21, of a first and second lever configured to receive a resistance unit having a compound aperture thereon.

FIG. 23 is a cross-sectional view through the assembly of FIG. 22.

FIG. 24 is an elevational view of the embodiment of FIG. 22, assembled but without a resistance element.

FIG. 25 is a posterior elevational view of a human pelvis, showing the axis of AP plane rotation relative to the iliac crest and a right side resistance unit of the present invention in an as worn orientation.

FIG. 26 is a side elevational view of a force transfer assembly have a “V” configuration.

FIG. 27 is a side elevational view of a force transfer assembly having an adjustable docking station.

FIG. 28 is a detail view of the docking station of FIG. 27.

FIG. 29 is a side elevational view of the force transfer assembly of FIG. 27, having a resistance unit mounted thereon.

FIG. 29A is a cross section taken along the line 29 A-29 A in FIG. 28, of a dock support having two degrees of freedom.

FIG. 29B is a cross section taken along the line 29 A-29 A in FIG. 28, of an alternative configuration restricted to one degree of freedom.

FIG. 29C is a perspective view of a multi axial force transfer docking assembly.

FIG. 29D is a side elevational cross sectional view of the assembly of FIG. 29C.

FIG. 29E illustrates the assembly of FIG. 29C having a power module mounted thereon and oriented as a left side mounted unit at about heel strike femoral extension.

FIG. 30 is a side elevational view of a resistance harness in accordance with the present invention.

FIG. 31 is in enlarged perspective view of a rotary damper resistance unit useful in the present invention.

FIG. 32 is a perspective view of the rotary damper of FIG. 30, with a portion of the housing removed to reveal a rotational resistance subassembly and an electronically enabled subassembly.

FIG. 32A is an exploded view of a resistance unit and an interchangeable electronic module.

FIG. 32B schematically illustrates a rotary encoder that can be integrated into the resistance device or power module of the present invention.

FIG. 33 is a side elevational view of a garment having a modular resistance unit interacting with four sensors to measure force or proximity to determine power exerted and/or calories burned.

FIG. 34 is a block diagram of sensor electronics, which may be carried within or attached to the resistance unit housing.

FIG. 35 is a block diagram of a remote display unit.

FIG. 36A is a block diagram of a bilateral power measurement system.

FIG. 36B illustrates an electronic computing environment according to an embodiment of the present disclosure.

FIG. 36C illustrates a block diagram of a parameter processing system including example inputs and outputs according to an embodiment of the present disclosure.

FIG. 37 shows torque as a function of angular velocity (expressed as RPM) for three resistance elements in accordance with the present invention.

FIG. 38 shows hip flexion and extension angle throughout a stride, relative to the pelvis.

FIG. 39 shows hip flexion and extension angle throughout a stride, relative to a vertical.

FIG. 40 is a graph depicting how a leg rotation changes with respect to time in an embodiment.

FIG. 41 is a graph depicting how rotational velocity in RPM of a leg changes with respect to time in an embodiment.

FIG. 42 is a graph depicting how power generated by rotation of a leg changes with respect to time in an embodiment.

FIG. 43 is a graph depicting how power generated by viscous damper type resistance units and rotation of a leg changes with respect to time in an embodiment.

FIG. 44 is a graph depicting cumulative power generated by viscous damper type resistance units with respect to time in an embodiment.

FIG. 45 is a flow chart describing steps to calculate rotational velocity of a leg, torque of viscous damper type resistance units, angular velocity, angular acceleration, power generated by rotation of a leg, and power generated by viscous damper type resistance units as performed in an embodiment.

FIG. 46 is a flow chart describing steps to calculate rotational velocity of a leg, torque of viscous damper type resistance units, angular velocity, angular acceleration, power generated by rotation of a leg, power generated from viscous damper type resistance units, power generated from wind resistance, and power generated from elevation as performed in an embodiment.

### DETAILED DESCRIPTION OF THE PREFERRED EMBODIMENT

Detailed descriptions of the preferred embodiments are provided herein. It is to be understood, however, that the present invention may be embodied in various other forms. Therefore, specific details disclosed herein are not to be interpreted as limiting, but rather as a basis for the claims and as a representative basis for teaching one skilled in the art to employ the present invention in virtually any appropriately detailed system, structure or manner.

In general, the devices in accordance with the present invention are designed to provide resistance to motion between a first region and a second region of the body such as across a simple or complex joint, (e.g., hip, knee, shoulder, elbow, etc.), throughout an angular range of motion. The resistance can be either unidirectional, to isolate a single muscle or muscle group, or preferably bidirectional to exercise opposing muscle pairs or muscle groups. Optionally, the device will be user adjustable or interchangeable to select uni or bidirectional resistance, and/or different resistance levels.

The devices of the present invention also or alternatively monitor any of a variety of biometrics, including power expended at the hip or incremental power expended as a result of the resistance.

The specific levels of resistance will vary depending upon the targeted muscle group, and typically also between flexion and extension across the same muscle group and the training or toning goal. Also wearer to wearer customization can be accomplished, to accommodate different training objectives. In general, resistances of at least about 10, and often at least about 15 or 18 or 20 or more inch-pounds will be used in heavy toning or strength building applications on both flexion and extension. All torque ratings described herein represent the torque measured at 40 degrees per second, which is an angular velocity that approximates walking.

Toning garments intended for long term wear or lighter toning may have lower resistance, with extension normally equal to or greater than flexion. Torque provided by a resistance element intended for the hip for toning garments may be at least about 4 in-lbs., sometimes at least about 6 or 8 or 10 or more in-lbs. depending upon the desired result, measured at 40 degrees per second. Torque will typically be less than about 20 in-lbs., and often less than about 16 or 14 in-lbs. In some implementations, torque will be within the range of from about 2-5 in-lbs for a ‘light’ toning element; within the range of from about 5-8 in-lbs for a ‘medium’ toning element; and within the range of from about 8-12 or 15 in-lbs for a ‘heavy’ toning element.

Devices specifically configured for rehabilitation (following stroke, traumatic injury or surgical procedure) may have the same or lower threshold values as desired.

Resistance experienced by the wearer is generated by a resistance element having a housing and a lever rotatable about a pivot point with respect to the housing. Rotation of the lever with respect to the housing encounters a preset level of rotational resistance generated by the internal operation of the resistance element.

The lever is secured within the leg of the garment so that it moves with the wearer's leg throughout the stride relative to a pivot point on the upper, lateral side of the hip. During a normal stride, the femur rotates about a transverse axis of rotation which extends from side to side through the approximately spherical right and left femoral heads, as they rotate within the corresponding right and left complementary acetabular cups in the pelvis. The pivot point on each of the right and left sides of the garment aligns approximately with that natural axis of rotation.

A connector is attached to the garment approximately at the pivot point and secured to prevent rotation of the connector. As long as the connector is restrained from rotating relative to the wearer's waist, the wearer will experience resistance imparted by the resistance element throughout the stride cycle. However, if the resistance exceeds a predetermined rating for a given garment, torque from the wearer's stride may cause the connector to rotate, by stretching the fabric in a twisting pattern concentrically about the axis of rotation. Twisting of the connector about its axis will absorb torque generated by the resistance element, thereby reducing the resistance perceived by the wearer, and the effectiveness of the system.

In view of the foregoing, the connector is secured with respect to the garment in a manner that will not permit it to rotate during use of a resistance element for which the garment is rated. Thus, there is an interplay between the stretch of the garment, the maximum anticipated torque applied by the wearer, and the manner in which the resistance element is secured to the garment. A connector mounted on a non-stretch garment, a garment fabricated with non-stretch panels or straps, or a harness constructed with non-stretch materials may be able to function under substantial applied loads without failure. Garments with higher stretch fabric and/or lower tensile strength to failure levels will only support relatively lower applied torque levels, unless supplemented with lower stretch filaments, lower stretch fabrics or other reinforcement straps or materials as will be appreciated by those of skill in the art.

In general, a garment ‘failure’ point is considered to have been achieved when the amount of rotational torque applied to the connector will rotate the connector (by stretching/deforming the garment) at least about 15 degrees, while the garment is being worn by a person or equivalent three dimensional fixture that stretches the garment within the range intended by the manufacturer (the garment is of the appropriate size for the wearer or fixture). Preferably, the connector will rotate no more than about 10 degrees, or no more than about 5 degrees, or optimally no more than about 3 degrees upon application of the maximum rated torque for that garment.

A light weight toning garment, for example, depending upon the garment stretch characteristics, may be able to withstand application of at least about 6 or 8 or 10 inch pounds of torque, before rotation of the connector through an angle of 5 degrees or other specified rating. A higher resistance garment may be able to withstand application of at least about 10 or 12 or 14 inch pounds of torque, before exceeding its rating. More athletic garments or harnesses, with woven nylon or leather straps for example, can be configured to withstand applied torques of at least about 20 or 25 or 30 or more inch pounds, depending upon the intended performance. Optimization of the foregoing variables for a particular product can be accomplished by those of skill in the art in view of the disclosure herein, to obtain a garment and resistance unit pairing that meet the desired performance characteristics.

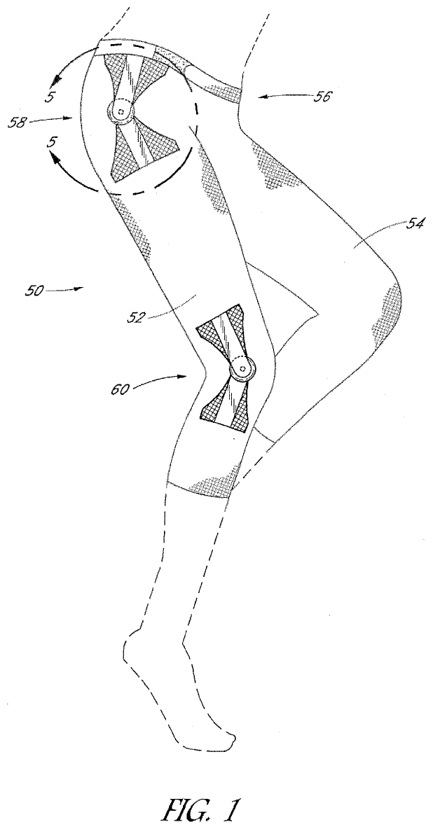

Referring to FIG. 1, there is illustrated a toning garment 50 in accordance with the present invention. The toning garment 50 includes a right leg 52, a left leg 54, and a waist 56. As for all garments disclosed herein, the toning garment 50 will preferably be bilaterally symmetrical. Accordingly, only a single side will be discussed in detail herein.

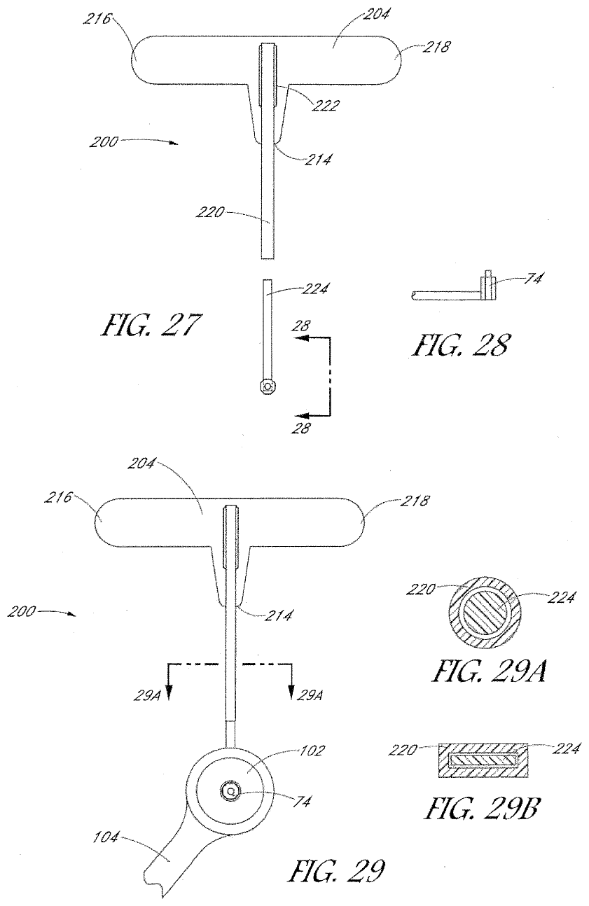

In the illustrated embodiment, the right leg 52 is provided with a hip resistance unit 58. Right leg 52 is additionally provided with a knee resistance unit 60. Each leg of the toning garment 50 may be provided with either the hip resistance unit 58 or the knee resistance unit 60, with or without the other. The left and right hip resistance units will preferably have an axis of rotation that is functionally aligned with a transverse axis of rotation which extends through the wearer's left and right hip axes of rotation. See, e.g., FIG. 25. Functional alignment includes precise alignment (coaxial) however due to the different fit that will be achieved from wearer to wearer, precise alignment may not always occur. Due to the stretchability of the garment, minor misalignment may self correct or not present adverse performance. Similarly, the knee resistance units, if present, will preferably have an axis of rotation that is functionally aligned with the transverse axis of rotation that extends through the center of rotation of each knee.

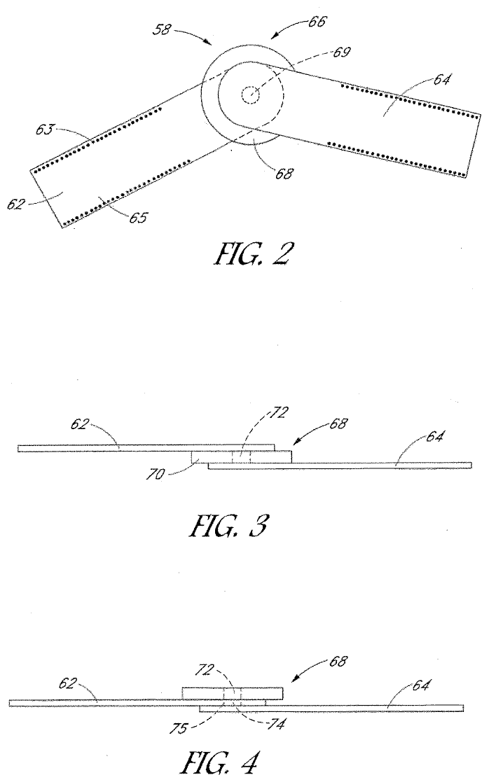

Referring to FIG. 2, the hip resistance unit 58 will be described in further detail. The left and right hip resistance units, and both the right and left leg knee resistance unit 60 may be constructed in a similar manner although may impart different torque levels.

The hip resistance unit 58 is provided with a first attachment such as a first lever 62, and a second attachment such as a second lever 64 connected by a pivotable connection 66. The pivotable connection 66 comprises a resistance element 68 which provides resistance to angular movement between a primary longitudinal axis of first lever 62 and a primary longitudinal axis of second lever 64. In the as worn orientation, the axis of rotation 69 is preferably substantially aligned with an axis of rotation of the joint with which the resistance element is associated.

A lever as used herein refers to a structure that mechanically links a docking plate, connector, housing or resistance element to a portion of the garment or wearer at or above or below the resistance unit, so that movement of the wearer is resisted by the resistance unit and applies a torque to the point of attachment to the garment without undesirable stretching or wrinkling of the garment. The lever may take a conventional form, as illustrated in FIG. 2, and comprise an elongate element having a length generally at least about 2 inches, in some embodiments at least about 4 or 6 or 8 inches to provide better leverage and attachment force distribution. The element may a have a width of at least about 0.25 inches, and in some embodiments at least about 0.5 inches or 1.0 inches or 2 inches or more but normally less than about 3 inches or 2.5 inches. The thickness may be less than about 0.25 inches, preferably less than about 0.125 inches and in some embodiments less than about 0.050 inches to maintain a low profile that can be concealed within or underneath the fabric of the garment. The lever may comprise a two part telescoping element, with a rod axially movably carried by a support such as a tube, as is discussed further below. The lever may comprise any of a variety of washable, non-corrosive materials such as nylon, Teflon, polyethylene, PEBAX, PEEK or others known in the art. Preferably the lever arm has sufficient structural integrity to transmit force in the anterior—posterior direction in the case of hip and knee resistance units, but is flexible in the medial—lateral direction to enable the garment to follow the contours of the body. See, e.g., FIG. 25.

The inferior and superior lever arms may be similar to each other for a resistance unit mounted at the knee. For a resistance unit mounted at the hip, the lever arms may be distinct. For example, the inferior lever arm at the hip may conveniently comprise an elongated femoral lever, such as that illustrated in FIG. 1 or 16, in which the axial length of the lever is at least about two times, and may be at least about three times or five times its width. This lever arm can extend down the lateral side of the leg, secured by the garment approximately parallel to the femur.

The superior lever arm may have a vertical component extending upward in the coronal plane towards the waist, with a bend or “T” so that a superior component extends in a transverse direction, either partially or completely circumferentially around the waist of the wearer. The transverse component may comprise a stretch fabric or relatively inelastic belt with a buckle or fastener. The superior lever may take the form of a “V” with the connector at the bottom (apex) of the V and the legs of the V stitched or otherwise bonded to the waist.

Alternatively, the superior lever arm may comprise a fabric, polymeric, or metal (e.g. Nitinol mesh) force transfer patch, such as a circular, square, rectangular, oval, “T” or other shape which can be secured to the rotational damper or a docking station for receiving the rotational damper, and also secured to the garment or the wearer or formed as an integral part of the garment, in a manner that resists rotation of the damper with respect to the garment during movement of the inferior lever. Thus, “lever” as used herein is a force transfer structure which resists rotation of the dock and is not limited to the species of a conventional elongate arm.

Either the superior or inferior lever may comprise a docketing platform for attachment to the resistance unit, and a plurality of two or three or four or more legs such as straps that are secured such as by stitching or adhesive bonding to the garment. See FIG. 8 in which a dock 80 supports at least an anterior element 82, a medial element 84 and a posterior element 86. Each of the elements is preferably relatively inflexible in the anterior-posterior direction, but flexible in the medial—lateral direction to enable the anterior element 82 to wrap at least partially around the side and optionally around the front of the leg. The posterior element 86 preferably wraps at least partially around the posterior side of the leg. The lever elements can be configured as a system of straps. The elements can comprise one or more strands or technical fabric supports, sufficient to transmit the forces involved in a given garment and resistance unit system.

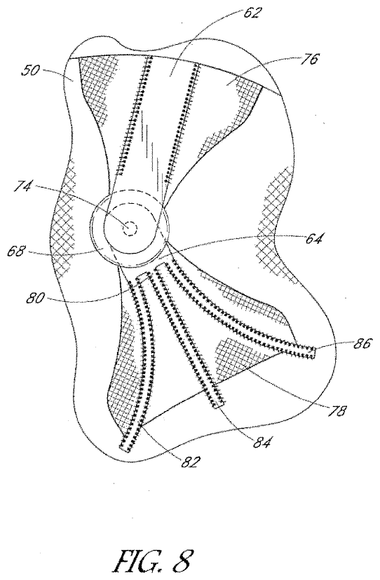

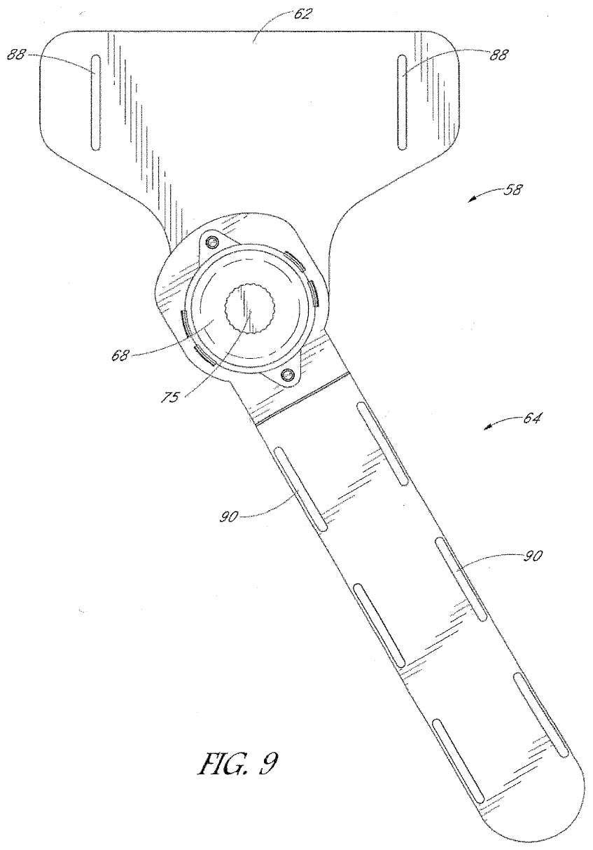

The hip resistance unit 58 may be secured to the toning garment 50 in any of a variety of ways. Referring to FIGS. 2 and 5, the first lever 62 is provided with at least a first set of apertures 63 and optionally a second set of apertures 65 to receive a filament such as a polymeric or fabric thread, for sewing the hip resistance unit 58 to the garment. Stitching may alternatively be accomplished by piercing the first lever 62 directly with the sewing needle, without the need for apertures 63 or 65. Alternatively, the first lever 62 can be secured to the garment using any of a variety of fastening techniques, such as adhesive bonding, grommets or others known in the art.

Since torque equals force times radius or length, a lever is convenient to distribute force to the garment. The inferior lever can extend inferiorly along the coronal plane, along a portion of the length of the femur. The longitudinal axis of the first, superior attachment at the hip may be transverse to the longitudinal axis of the second lever 64 at the midpoint of its range of motion, such that the first lever is aligned like a belt, circumferentially extending along a portion of or approximately parallel to the wearer's waist displaced superiorly from the axis of rotation of the wearer's hip. Normally the hip axis of rotation will be offset inferiorly by at least about 3 inches, and often 5 inches or more from the iliac crest, which approximates the top of the belt line for many wearers. Alternatively, the housing of the resistance element or docking platform may be sewn or adhesively bonded or otherwise attached directly to reinforced fabric at the hip such as by circular weaving or stitching techniques known in the art.

The resistance element 68 may be any of the resistance elements disclosed in U.S. patent application Ser. No. 14/665,947 filed Mar. 23, 2015, now published as U.S. 2015/0190669, the disclosure of which is hereby incorporated by reference in its entirety herein. In one embodiment, resistance element 68 may comprise a rotary damper containing a fluid such as air, water or a viscous media such as silicone oil. The rotary damper may be rated to provide anywhere within the range of from about 0.1 inch pounds to about 50 inch pounds torque at a rotational velocity of 40 degrees per second depending upon the joint or other motion segment to be loaded and desired intensity. Typical torque ranges are disclosed elsewhere herein.

Resistance imposed at the knee will generally be less than at the hip. Values of generally no more than about 85% or 50% or 35% of the torque at the hip may be desirable in a toning garment at the knee, measured at 40 degrees per second. As discussed elsewhere herein, the resistance element at any given joint can provide the same or different resistance (including zero) upon flexion or extension.

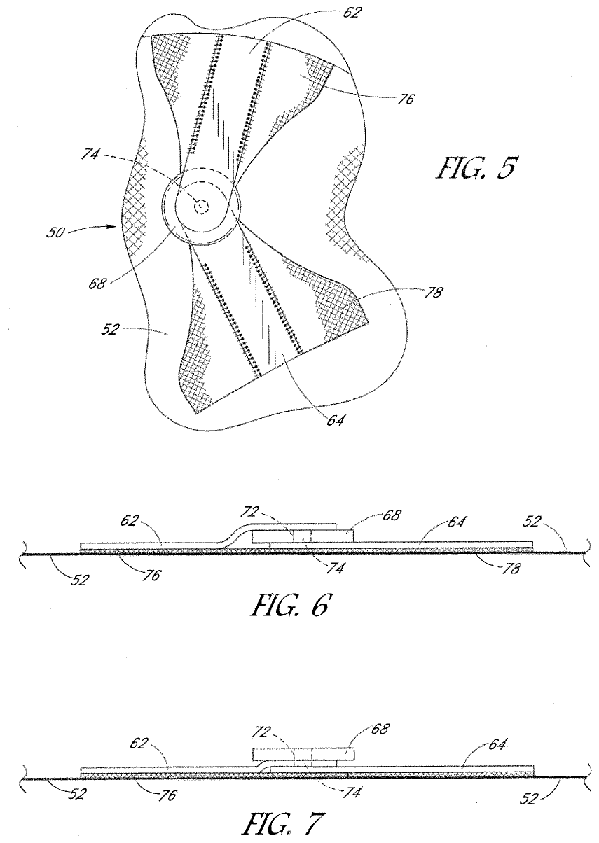

Referring to FIGS. 3-4, the resistance element 68 may comprise a generally disc shaped housing, having a diameter of less than about 4 or 3 or 2.5 inches, and a thickness in an axial direction of less than about 0.75 and preferably less than about 0.5 inches. A connector 72 is rotatably carried by the housing 70. Connector 72 may be a post or an aperture, having a non-circular (e.g. square, hexagonal, triangular, circular with at least one spline or flat side) keyed cross-section such that a complementary post or aperture may be axially positioned in engagement with the connector 72, to transmit rotational torque.

Referring to FIGS. 3-4, the resistance element 68 housing 70 may be secured to either the first lever 62 or the second lever 64 or neither, as is described below. The connector 72 may be secured to the other of the first lever 62 and second lever 64. Resistance element 68 thus provides resistance to motion of the first lever 62 with respect to the second lever 64, throughout an angular range of motion about the axis of rotation 70.

In an alternative configuration, the levers may be mounted on the same side of the resistance element 68 to provide an overall lower profile. Referring to FIG. 4, second lever 64 is provided with a connector 72 in the form of a post for rotationally engaging the connector on resistance element 68 which is in the form of a complementary aperture. Post 74 extends through an aperture 75 in the first lever 62. Aperture 75 has a diameter that exceeds the maximum transverse dimension of the post 74, such that post 74 may rotate without imposing any force on first lever 62. The housing of resistance element 68 is immovably secured with respect to first lever 62 such as by adhesive bonding, molding, interference snap fit or other immovable connection.

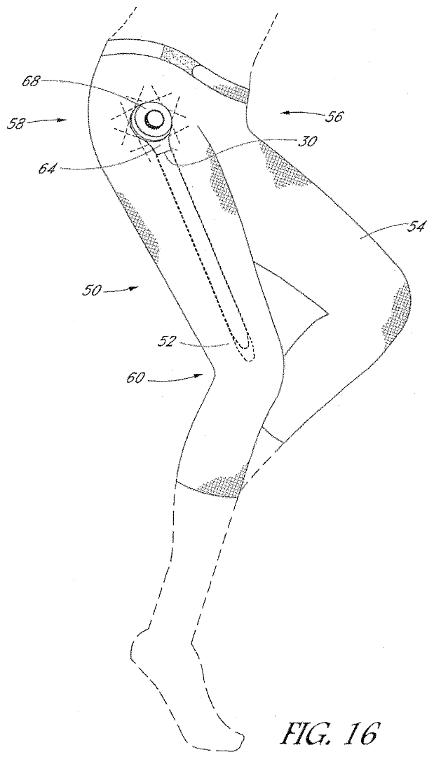

Referring to FIG. 5, a hip or knee resistance unit 68 is illustrated as secured to a garment 50 although the following description also applies to resistance elements at the elbow, wrist, ankle or knee. Depending upon the configuration of the lever arms, the stretchability of the fabric, and the level of resistance imposed by resistance element 68, one or more reinforcement or force transfer or dissipation features may be necessary to transfer sufficient force between the lever arm and the garment, while minimizing stretching or wrinkling of the garment. In the illustrated embodiment, first lever 62 is additionally provided with a first force dissipation layer 76. Force dissipation layer 76 may comprise any of a variety of meshes or fabrics, such as those disclosed previously in US 2015/0190669 which is hereby incorporated in its entirety herein by reference, and below in connection with FIG. 14.

In one implementation, the fabric comprises one or more strands of yarn or filament 77 having a vector extending in the as worn anterior posterior direction which exhibits relatively low stretch. See FIG. 14. A plurality of strands 77 can be woven in an orientation that is approximately at a tangent to at least about 2 or 4 or 8 or 10 or more points on a concentric circle around the rotational axis of the resistance element or force transfer layer to optimize resistance to rotation of the housing relative to the garment. Force dissipation layer 76 may be attached to the edges and/or lateral and/or medial surfaces of first lever 62 or the damper housing or docking platform for receiving a damper such as by stitching, adhesives or other fastener, and extend in the anterior posterior direction beyond the edges of the first lever 62 to provide an attachment zone both anteriorly and posteriorly of the first lever 62. In the embodiment of FIG. 14, the force dissipation layer is the lever, securing the damper against rotation with respect to the adjacent fabric overlying the axis of rotation. The attachment zones may be secured to the underlying garment by stitching, adhesives or both, or straps, strands or other fasteners known in the art.

The first force dissipation layer 76 may extend beneath, within the same plane, or across the outside (lateral) surface of the first lever 62, entrapping the first lever 62 between the force dissipation layer 76 and the garment 50. Alternatively, the force transfer layer may function as a lever.

The force dissipation layer (whether an overlay or the actual sidewall of the garment) may be molded mesh or a technical fabric weave, comprising any of a variety of strands identified in US 2015/0190669 previously incorporated by reference herein. Preferably the fabric has stretch resistance along at least one axis, which can be aligned with an axis under tension during flexion or extension due to the resistance element (e.g. the AP plane). The fabric may exhibit a higher level of stretch along other axes. The fabric also preferably exhibits low weight, high breathability and high flexibility. Some suitable fabrics include shoe upper fabric from running shoes including, for example, that disclosed in US patent publication No. 2014/0173934 to Bell, the disclosure of which is incorporated by reference in its entirety herein. Additional multilayer fabrics having good flexibility, and stretch resistance along one axis and higher stretch along a transverse or nonparallel axis, useful for the force dissipation layer are disclosed in U.S. Pat. No. 8,555,415 to Brandstreet et al; U.S. Pat. No. 8,312,646 to Meschter et al; and U.S. Pat. No. 7,849,518 to Moore et al., the disclosures of each of which are incorporated in their entireties herein by reference. Typically, the force transfer layer will have lower stretch along at least one axis than the stretch of the underlying garment.

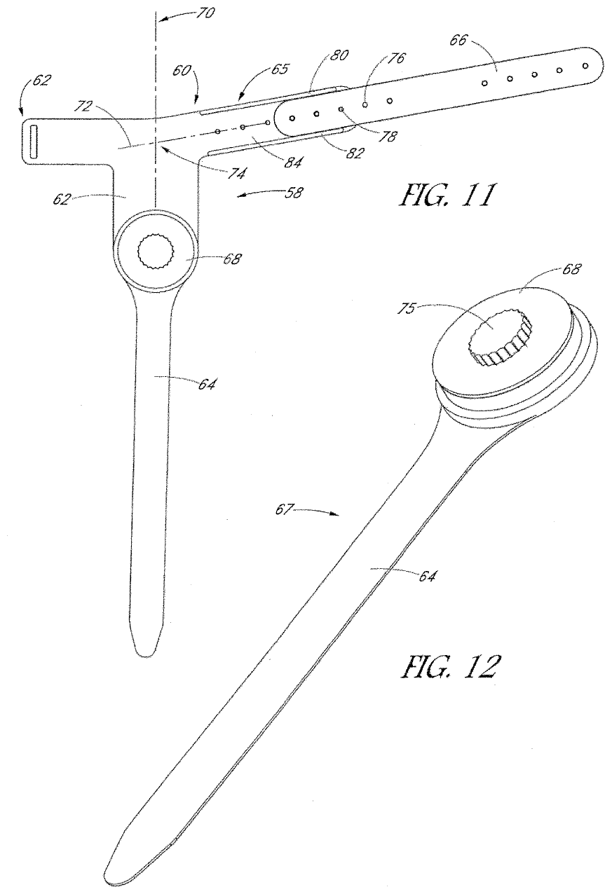

Referring to FIG. 9, there is illustrated a resistance unit 58 comprising a first lever 62 configured for attachment to the garment or to the wearer to at least approximately align the rotational axis of the resistance element with the hip, as discussed below. First lever 62 may be provided with any of a variety of attachment structures such as a force dissipation layer, straps, Velcro or at least one and typically two or more slots, snaps or other attachments 88 for connection to a strap, belt or other fastener associated with the garment. First lever 62 may comprise any of a variety of polymeric or metal sheets or mesh membranes, printed, molded or machined parts or fabrics disclosed elsewhere herein, which may be bonded or stitched directly to the garment, or held by a belt to the outside of the garment.

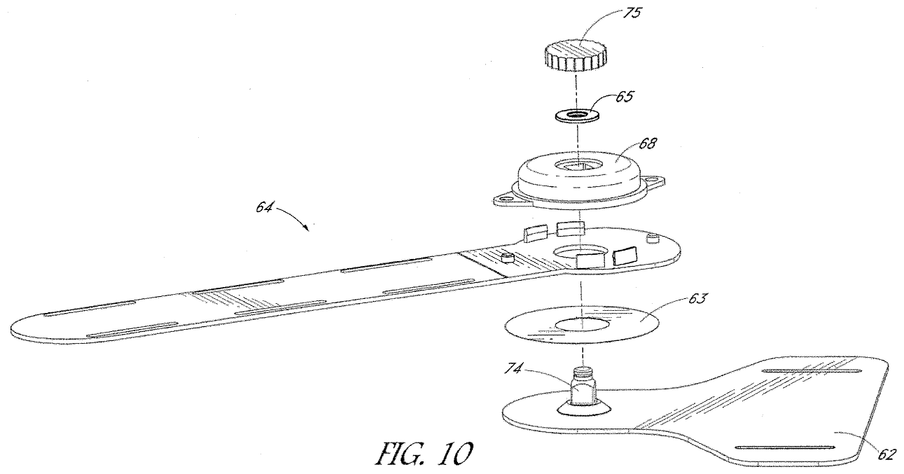

Lever 62 is pivotably connected to a second lever 64 by way of resistance element 68 as has been described. Resistance element 68 may comprise any of a variety of resistance elements, such as friction brakes, malleable materials, clutches, or rotary viscous dampers as has been discussed. Resistance element 68 may be securely permanently or removably mounted to the second lever arm 64 (as illustrated) or to first lever arm 62 or both. A post 74 (FIG. 7) is secured to the first lever arm 62, and extends through a complementary aperture in the resistance element 68. In this manner, rotation of the second lever 64 about the rotational axis of resistance element 68 with respect to the first lever 62 experiences the resistance provided by resistance element 68. Second lever 64 may be provided with a force dissipation layer and/or one or two or three or four or more inferior connectors 90. As illustrated, inferior connectors 90 may be apertures such as slots for receiving a strap or filament for securement to the pant leg or the leg of the wearer.

Preferably, a quick release 75 is provided, to engage and disengage the resistance element, and or enable disassembly into component parts. Quick release 75 is illustrated as a knob which may be rotatable, or axially movable between a first and a second position to engage or disengage the damper. Any of a variety of quick release mechanisms maybe utilized, such as a threaded engagement, or a pin or flange which can rotate into engagement behind a corresponding flange or slot. Quick release 75 allows rapid removal of the damper, or the damper and femoral lever arm, as is discussed in more detail below.

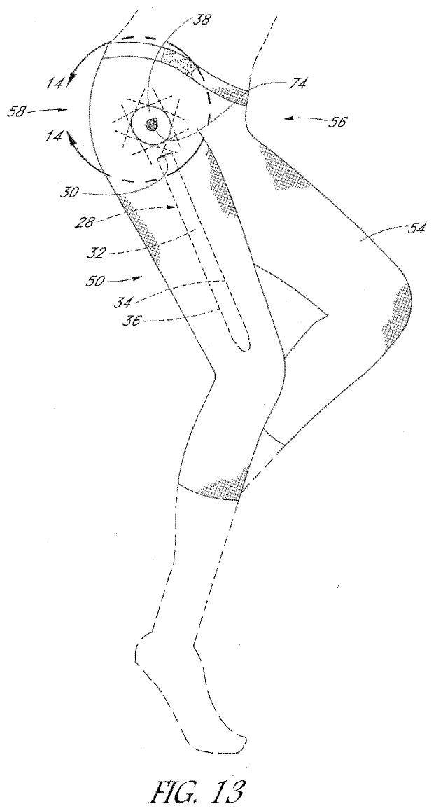

Referring to FIG. 10, an exploded view illustrates the first lever 62 having post 74 secured thereto such that rotation of the post is transferred to the lever 62. A friction modifier 63 such as a washer or membrane that may comprise a friction reducing material such as a lubricious polymer (e.g., PTFE) may be provided to separate the first lever 62 from second level 64. Alternatively the friction modifier 63 may be a friction enhancer, such as one or two or more washers having a friction enhancing surface texture, which create resistance to movement and can therefore supplement or replace the rotational damper.

Connectors 65 may be provided for locking the construct together. Connectors 65 may comprise one or more locking rings, nuts, pins or other structure. Preferably, a quick release mechanism 75 such as a quick release lever, rotatable knob or snap fit that allows the wearer to quickly engage or disengage the resistance unit 58 into component subassemblies, as will be described.

Skeletal motion at the hip during normal activities including walking involves complex, multidirectional movement of the femoral head within the acetabular cup. However when viewed to isolate out the single component of movement in the anterior-posterior (“AP”) plane, the femur swings forward and back like a pendulum, pivoting about a rotational axis 69 (FIG. 25) which extends laterally through the approximate centers of the roughly spherical left and right femoral head.

Many of the resistance elements disclosed herein exhibit a fixed axis of rotation. Ideally, the exercise garment of the present invention of the type having a fixed rotational axis can be worn by a wearer such that the rotational axis of the resistance element is coincident with the rotational axis 69 of the femur. However, due to a combination of factors including the stretch of the fabric and dissimilarities from wearer to wearer in the contour of the soft tissue between the femur and the garment, the two rotational axes may not perfectly align. An imaginary straight-line in the AP plane which connects the anatomical rotational axis and the rotational axis of the resistance element defines a non-zero offset in the case of misalignment between the two axes of rotation which has the effect of a piston like pulling or pushing the second lever 64 along its longitudinal axis relative to the femur throughout the stride cycle. If force in all directions from the second lever 64 is effectively transmitted to the garment, this axial reciprocal movement of the second level 64 with respect to the wearer and garment through the offset distance 26 may cause a variety of undesirable results, including chafing of the garment up and down against the leg, wrinkling, buckling or damaging the fabric of the garment and/or the material of the second lever 64.

It may therefore be desirable to decouple axial movement of the second lever 64 from the garment, while maintaining a high degree of force transmission between the second lever 64 and the garment in the AP plane.

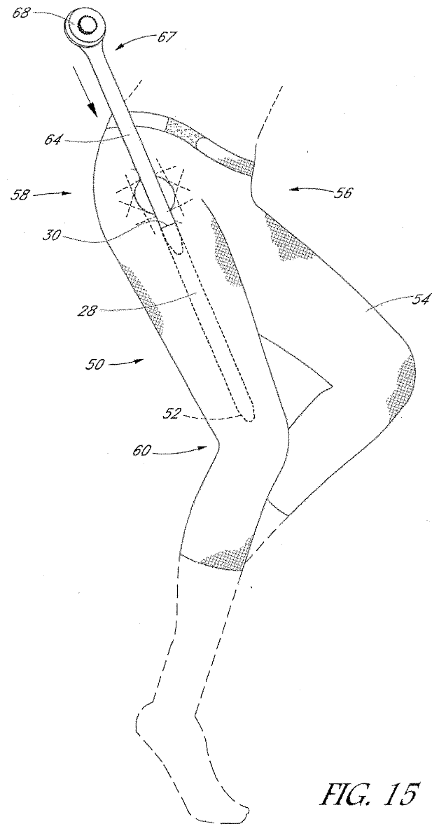

Referring to FIG. 13, one convenient structure for accomplishing the foregoing is to provide an elongated pocket 28 extending in an inferior superior direction along the lateral side of each leg of the garment. The pocket 28 comprises an opening 30 at a superior end thereof, providing access to an elongate cavity, for removably receiving the second lever 64. An anterior limit 34 of the pocket 28 and a posterior limit 36 of the pocket 28 are dimensioned relative to the width of the second lever 64 to provide a snug fit against relative AP movement, but which permits axial sliding of the second lever 64 along its longitudinal axis within the pocket. The axial length of the pocket exceeds the axial length of the second level 64, thereby enabling the second level 64 to reciprocate up and down within the pocket 28 without transmitting inferior superior axis movement to the garment.

The axial length of the pocket 28 is preferably at least about 4 inches, and in some implementations it is at least about 6 inches or 8 inches or more in length, depending upon the garment size, fabric stretch and resistance level of the resistance unit. The length of the pocket will preferably exceed the length of the associated lever by an amount sufficient to compensate for the likely offset between the rotational axis of the hip and the rotational axis of the damper. Typically, that offset will be no more than about 2 inches, and preferably no more than about 1 inch or 0.5 inches.

The lever 64 will preferably axially reciprocate within the pocket 28 with minimal friction. For this purpose, the lever may be constructed from or coated with a lubricious material. In addition, the interior surface of the pocket preferably comprises a material with a low coefficient of friction with respect to the surface of the lever. The interior of the pocket 28 may be provided with one or two or five or 10 or more axially extending filaments or raised ridges, to reduce the contact surface area between the lever 64 and the pocket 28. The interior of the pocket 28 may be lined either partially or completely with a membrane having a low friction surface. Thus, a pocket liner comprising any of a variety of materials such as nylon, PTFE, polyethylene terephthalate, PEEK, metal films or other materials may be utilized depending upon the intended performance characteristics.

The inside width of the pocket is preferably dimensioned such that the lever is not able to move significantly in the AP plane with respect to the pocket. The width of the pocket with the lever installed therefore preferably only exceeds the width of the lever by a sufficient amount to permit the desired axial movement of the lever without transferring axial movement to the garment. The width may be adjustable between a larger width such as for inserting the lever, and a smaller width for efficient lateral force transfer. That may be accomplished by fabricating the pocket from compression fabric so that it stretches to receive the lever. Alternatively, a zipper may be advanced along the length of the pocket to bring two parallel edges closer together, with straps connected to the pant leg on one side of the pocket and connectable (e.g., with Velcro) to the pant leg on an opposite side of the pocket.

Alternatively, the resistance unit 58 can be provided with any of a variety of axial expansion dampers, positioned between the rotational axis of resistance element 68 and a portion of the second lever 64 which is immovably secured to the garment. Axial extension dampers may include first and second side by side or concentric telescoping components, which through relative axial sliding motion allow the second lever 64 or other attachment point to the garment to reciprocally lengthen and shorten. See, e.g., FIGS. 27-29 discussed below. Alternative structures such as springs, collapsible diamond shaped cells, etc., can allow axial shortening and lengthening of the second lever 64 between the rotational axis and the point of attachment to the garment so that axial reciprocating movement of the femoral lever is not transmitted to the garment. The proximal end of the lever may be provided with an adjustable attachment element such as an elongate, axially extending slot which receives a complementary attachment element such as a post on the damper having two opposing flat sides so that the lever can reciprocate axially but remain rotationally keyed to the post.

Referring to FIG. 13, there is illustrated a garment having a docking station 38 for releasably receiving a resistance module 68. As illustrated in FIG. 14, the docking station 38 comprises a platform 42 for receiving a damper or other resistance module. The platform 42 comprises at least one connector 74, for connecting with the resistance module. The connector may be a post or an aperture, for keyed connection with a corresponding connector on the damper or other resistance module. The platform 42 or connector 74 may be provided with a quick release feature 44, for releasably engaging a complementary quick release control such as a lever, button or rotatable knob as has been discussed.

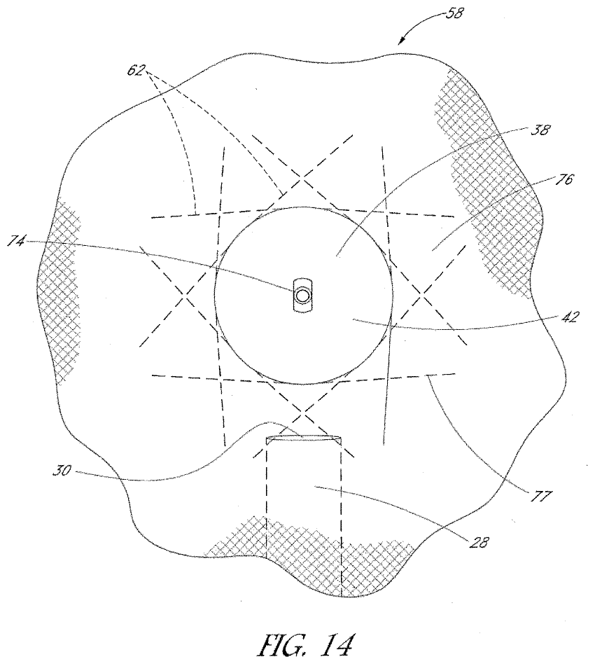

Referring to FIG. 11, there is illustrated a left side resistance unit 58 in the form of a harness or belt, or subassembly that can be attached to or integrated into a compression pant, athletic training short or pant, or other garment. The right side is omitted for clarity. The resistance unit 58 comprises a femoral lever 64 and a resistance element 68 as has been described. In this illustration, the first lever 62 is in the form of an approximately “T” or “Y” shaped hip support 60, configured to minimize the risk of rotation of the resistance element 68 with respect to the wearer. Hip support 60 comprises an anterior connector 62, such as a buckle or strap or other fastener for fastening across the anterior of the wearer's waist. The hip support 60 additionally comprises a posterior connector 65, for connection to or across the posterior side of the wearer or garment. In the illustrated embodiment, posterior connector 65 is adjustably connected to a posterior strap 66. The posterior strap 66 may be configured to extend across the posterior of the wearer and to connect to a right side resistance unit 58, such that the hip support 60 is connected to both the right and left resistance units 58, encircling at least a portion and preferably all of the waist of the wearer in the as worn configuration.

The axis of rotation of the resistance element 68 is displaced inferiorly from the wearer's waist line along an inferior—superior axis 70 by at least about 2 or 3 or 4 or more inches. The posterior connector 65 extends along a longitudinal axis 72 which intersects with the axis 70 at an angle 74. The angle 74 causes the axis 72 to deviate from perpendicular to axis 70 by at least about 2°, and in some embodiments at least about 3° or 5° or more.

The posterior strap 66 may be adjustably connected to the posterior connector 65. In one implementation, one of the posterior strap 66 or connector 65 is provided with a plurality of apertures 76. The other is provided with at least one post 78. In an alternate embodiment, the two components may be secured by Velcro, or a buckle. In a further implementation, the strap 66 is slidably engaged with the posterior connector 65. This may be accomplished, for example, by providing a first raised rail 80 and a second raised rail 82 defining a recess 84 there between within which the posterior strap 66 can slide. Posterior connector 65 may be retained within the recess 84 such as by a flange on one or both of the rails 80 and 82, or by connecting the rails 80 and 82 to form an enclosure for receiving posterior strap 66. Enclosure may be formed by a plastic restraint, integrally formed with the posterior connector 65, or by a fabric enclosure. Alternatively, the posterior strap 66 comprises a fabric or elastic such as a belt or waist band on a pant.

The components of the hip support 60 may comprise polymeric sheet or membranes, various technical fabrics as has been described elsewhere herein, or combinations of the two, in order to optimize comfort, fit and structural integrity of the connection of the hip support 62 to the wearer. Any portions or all of the hip support may be distinct structures attached to or worn over the top or under the garment, or may be structural fabric and components woven or sewn into the garment.

Preferably, the hip support 60 is constructed largely in fabric, such that it has sufficient flexibility and durability to be comfortable, durable, and able to withstand normal washing and drying cycles. In a preferred embodiment, the first lever 62 is provided with a docking station for removably receiving and engaging the resistance element 68 and second lever 64.

Thus, referring to FIG. 12, a modular detachable femoral resistance unit 67 may be provided. The femoral unit 67 may comprise one or both of the second lever 64 and the resistance element 68. In the illustrated embodiment, resistance element 68 is bonded or otherwise secured to or integrally molded with the second lever arm 64 to provide an integral modular femoral resistance unit 67.

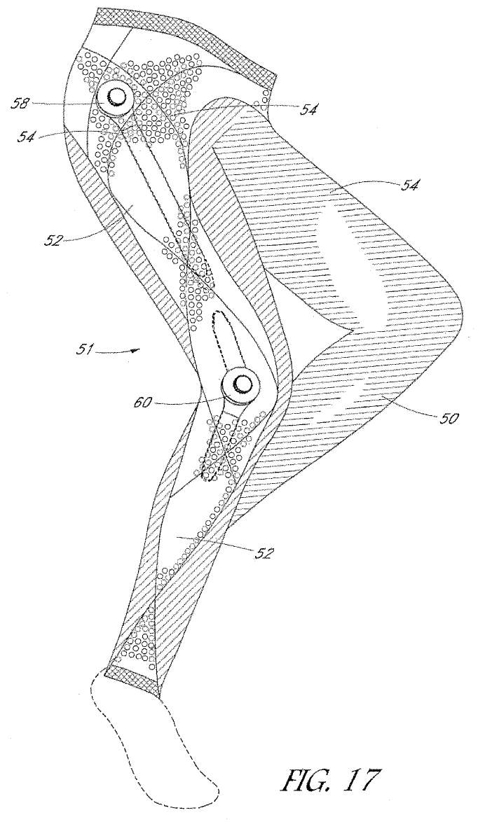

Referring to FIGS. 15 and 16, this configuration allows the wearer to put the garment on with just any of the hip docking platforms disclosed herein secured thereto. Once the garment is on, the second lever 64 may be inserted within the femoral attachment element such as pocket 28 running down the lateral side of the leg or otherwise removably secured to the garment or the wearer's leg. The resistance element 68 is then aligned with the docking platform on first lever 62, seated and coupled thereto. This may be accomplished by advancing a first connector such as the aperture on resistance element 68 over a second, complementary connector such as the post on first lever 62 to achieve rotational engagement, and locking the resistance element 68 into place using any of a variety of quick lock or release features. These include interference (snap) fit, or any of a variety of twist connectors, locking pins or levers or others known in the art.

The modular femoral resistance unit 67 may be uncoupled from the docking station such as by manipulating the quick release control, and removed from the garment to permit removing the garment from the wearer, and or placing the garment in the wash. In addition, a wearer may be provided with a plurality of matched pairs of modular femoral resistance units, each pair having matched resistance elements 68 with a different level of resistance from another pair. This modularity enables the wearer to select the desired level of resistance depending upon a given use environment, as well as to facilitate washing, and optimizing the useful life of whichever components of the detachable component resistance toning system have the greatest useful life. Additional details of suitable resistance elements are disclosed in US 2015/0190669, previously incorporated by reference herein.

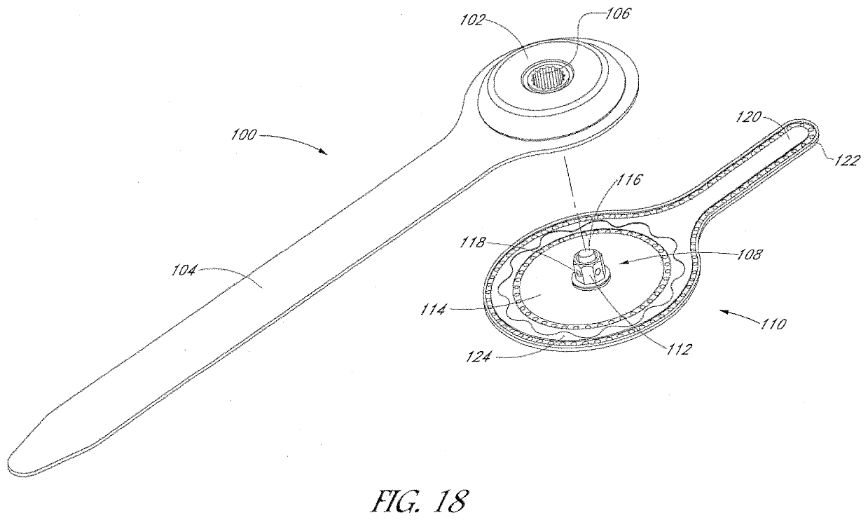

Referring to FIG. 17, at least one and in some implementations at least two or three or more technical fabric support panels 52 are provided on each of the right and left legs, to facilitate force transfer between the wearer and the hip resistance unit 58 and, when present, the knee resistance unit 60. The technical support panel 52 may be provided with at least one and normally a plurality of reinforcement strands 54 extending along a pattern to facilitate force transfer and maintaining fit of the garment throughout the range of motion in opposition to the resistance provided by the resistance unit. The technical fabric support panel 52 may be positioned over the entire height of the garment (as illustrated) or may be localized in the vicinity of the resistance units.

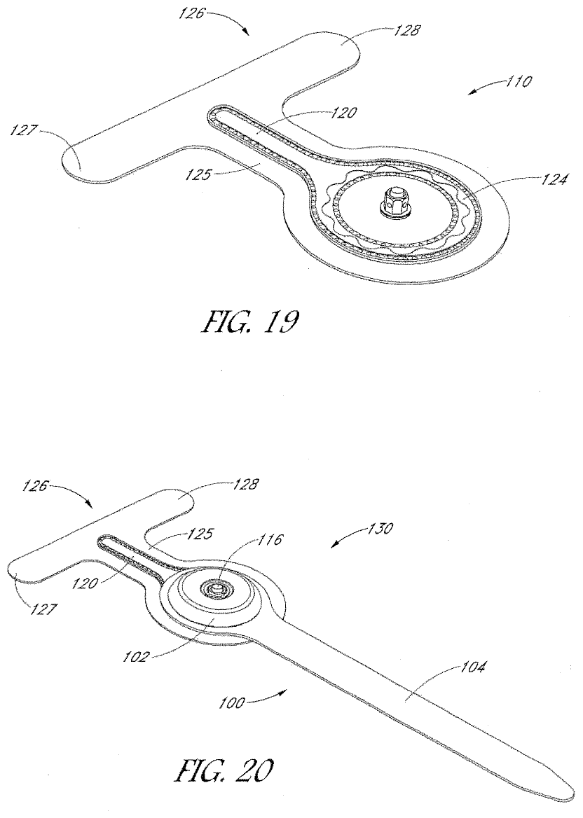

Referring to FIG. 18, there is illustrated an exploded perspective view of a first lever having a resistance unit thereon, and a complementary docking platform having a second lever. The resistance unit 100 comprises a resistance element 102 and a femoral lever 104. The resistance element 102 comprises a connector 106, which, in the illustrated embodiment, comprises an aperture.

The aperture is configured to receive a complimentary connector 108 such as a post 112 on the docking platform 110. The post 112 comprises at least one axially extending slot, flat side or other key to provide rotational interlock with a complementary surface structure on the connector 106. In the illustrated embodiment, post 112 comprises a polygon, such as a hexagon or octagon. Alternatively, the post 112 may have a cylindrical configuration and the complementary aperture comprises the aperture through a spring clutch on the resistance unit 100. A control such as a lever, slider switch or button may be carried by the housing of resistance element 102 to change the inside diameter of the aperture of the spring clutch as is understood in the art. The relative location of the complementary connectors can be reversed between the docking platform 110 and the resistance element 102 depending upon the desired product design.

Connector 108 is carried by a docking platform 110, which includes a base plate 114 secured to the post 112. Post 112 is provided with a quick release button 116, depression of which allows a plurality of interference locks such as a ball or post 118 to retract radially inwardly to disengage a complementary recess within the connector 106. Preferably, the connector 108 is not able to rotate with respect to plate 114.

In use, movement of the leg throughout a stride carries the femoral lever 104 through an arcuate path generally within the anterior posterior plane, which pivots about the axis of rotation extending through connector 108. The resistance unit transfers more or less rotational force to the post 112 depending upon the resistance rating of the resistance element 102. The docking platform 110 is configured to distribute rotational force transferred by the post 112 to a larger surface area of the underlying garment or to a point of greater distance from the axis of rotation to prevent the post 112 from rotating in a manner that twists or otherwise deforms the fabric of the compression garment.

Since the force applied to the garment at a given point is equal to the torque applied by the resistance element 102 during a stride times the radius or distance from the center of rotation to that point, a larger diameter docking platform 110 would more effectively distribute rotational force to the fabric without distortion. However, anatomical constraints due to the dynamic three dimensional configuration of the wearer and garment in the vicinity of the hip limit the diameter of the docking platform 110. Accordingly, one or more levers may extend radially outwardly or at a tangent or other angle to a circle concentric about the post 112 such as the best fit circle about the periphery of the docking platform 110.

In the illustrated embodiment, a lever 120 extends outwardly from the post 112 and docking platform 110 to increase the effective distance (radius) from the axis of rotation and better distribute rotational force. Lever 120 may extend at least about one or 2 inches from the periphery of the plate 114 or from the post 112 in an implementation where the plate is the same diameter as and/or an integral portion of the post 112 (effectively no distinct plate).

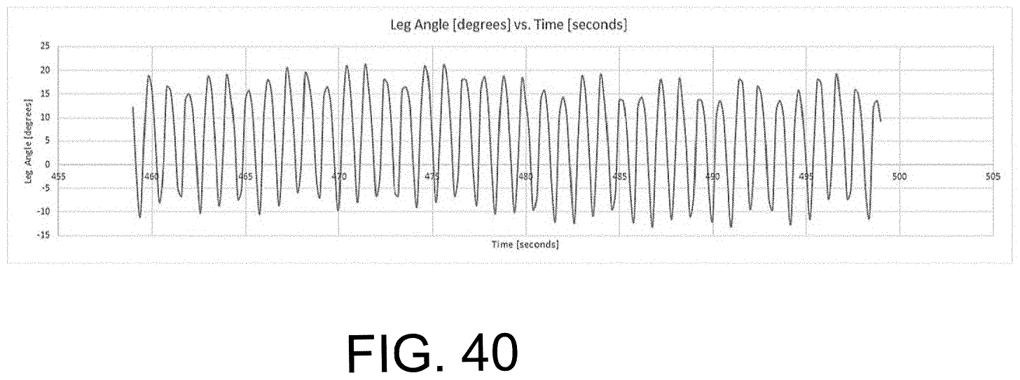

In some implementations, the lever 120 extends at least about four or 5 inches or more from the post 112. If the lever 120 is configured to reside on a coronal plane (approximately straight up and down) as illustrated, for example, in FIGS. 1 and 40, extending upwardly when the wearer is in a standing position, the lever will typically be no more than about 6 inches, but at least about 5 inches or 4 inches from the axis of rotation, depending upon the distance between the rotational axis of the hip and the top of the wearer's belt line. The superior lever 120 may alternatively extend circumferentially part way or all the way around the wearer's leg, or in a spiral or angled orientation inclining upwardly or downwardly from the post 112.

The docking platform 110 in the illustrated the embodiment is intended to be permanently secured to the garment. For this purpose, a plurality of apertures 122 may be provided at least around the periphery of the superior lever 120 and an interface 124 for connecting to the plate 114. In the illustrated embodiment, the interface 124 comprises a ring which may be integrally formed with superior lever 120. The ring includes an aperture for receiving the plate 114. To minimize the risk of rotation of the plate 114 within the ring, the inner diameter of the ring may have one or more rotational locking keys such as flat surfaces or radially facing projections or recesses such as the illustrated sinusoidal periphery, which interlocks with a complementary exterior circumference of the plate 114. Alternatively, the lever 120, plate 114 and optionally connector 108 may be integrally formed such as through molding or machining techniques known in the art.

At least one lever 120 and optionally two or more levers may be mechanically linked to the post 112, and the length of the lever or levers can be optimized based upon the stretch of the fabric of the underlying garment, along with the rated torque for the resistance unit 100 intended to be used with that garment.

FIG. 19 illustrates a docking platform 110 assembly as in FIG. 18, with the addition of a force transfer layer 125. As has been discussed, force transfer layer 125 is preferably a flexible fabric, molded mesh, metal mesh or other layer that provides a force transition between the superior lever 120 and the fabric of the garment. Force transfer layer 125 may be an integral part of the side wall of the garment, or may be an overlay, layered onto a garment.

In the illustrated embodiment, force transfer layer 125 extends outwardly beyond the periphery of the interface 124. This aspect of force transfer layer may be omitted. The most effective force transfer occurs at the superior end of superior lever 120, which is the greatest radius from the center of rotation. Thus, the force transfer layer 125 is preferably provided with a transverse band 126 which comprises or is attached to the waistband of the garment. Transverse band 126 may be provided with both a left strap 127 and right strap 128 which may each extend at least about 2 inches, and preferably at least about 4 inches or 6 inches or more from the midline of the superior lever 120. The transverse band 126 on the left resistance assembly may be connected with the transverse band 126 on a right resistance assembly either on the posterior side or the anterior side or both, of the wearer, to extend for a full circumference of the waist. In this configuration, the anterior connection between the left side and right side transverse bands is preferably provided with a releasable connector such as a buckle, or complementary hook and loop fastening straps for adjustable attachment to the wearer. The transverse band 126 may comprise a low stretch fabric or other material having sufficient structural integrity under tension that it resists movement of the superior lever 120 about the axis of rotation.

In one implementation of the invention, applicable to any of the embodiments described herein, the docking plate 114 is mounted with no direct attachment to the underlying garment. This allows the docking plate to float in response to anatomical movement, although not rotate relative to the axis of the post 112. The superior lever 120 will be securely attached to the garment, such as by transverse band 126 or other force transfer layer or attachment technique disclosed herein. Attachment may be constrained to an attachment zone within the upper 75%, upper 50%, upper 25% or less of the length of the superior lever, measured from the rotational axis. The attachment zone may extend inferiorly to the upper limit of the plate 114 or as far inferiorly as the level of the post 112. The remainder of the docking platform 110 below the attachment zone remains floating with respect to the garment. The upper lever 120 may be integrated into the garment or covered by a stretch panel and both the front and back sides remain unattached to the garment or cover layer outside of the attachment zone.

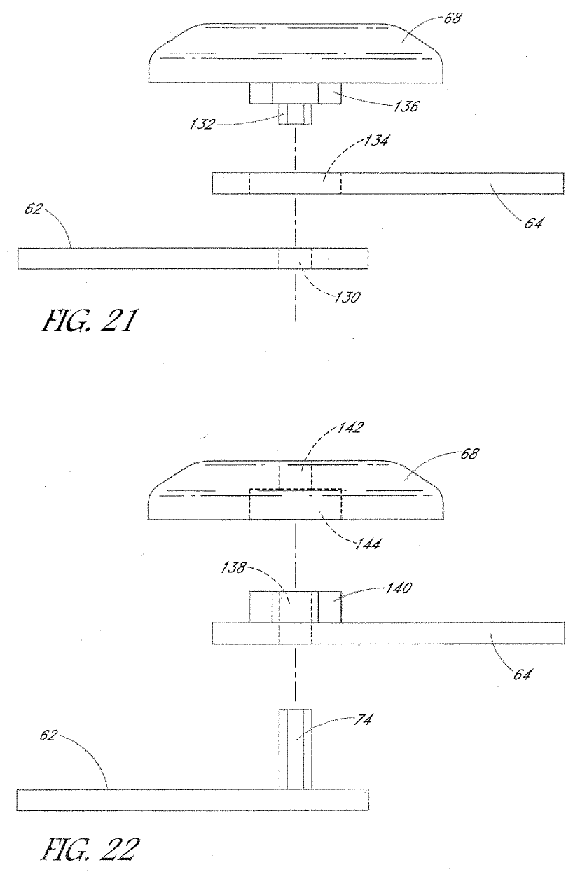

Referring to FIG. 20, there is illustrated a perspective view of a complete resistance subassembly 130, including an upper lever 120 attached to a force transfer layer 125 and a lower resistance unit 100 pivotably mounted on the docking station.

The modular resistance unit 100 has generally been illustrated as having a resistance element 102 mounted on a femoral lever 104. It may in some circumstances be desirable to allow the resistance element 102 to be removed from the garment as a separate unit, leaving both of the upper and lower levers permanently or removably coupled to the garment.

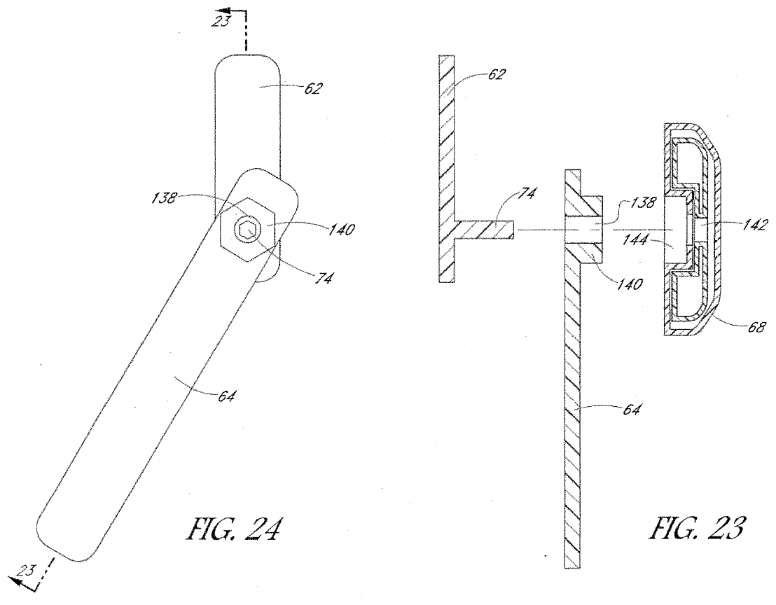

Referring to FIG. 21, there is illustrated an exploded view of a first lever 62 having a first aperture 130. A second lever 64 is provided with a second aperture 134. Both levers 62 and 64 may be permanently carried by the garment. Alternatively, either or both of the levers 62 and 64 may be removably carried by the garment.

When mounted on the garment, the first aperture 130 and second aperture 134 are substantially coaxial. First aperture 130 is provided with a keyed cross-section such that it receives a first complementary projection 132 on resistance unit 68 so that rotation of first lever 62 will cause an equal rotation of first projection 132. Keyed projections and complementary apertures may comprise at least one flat side or spline, and in some embodiments comprise a polygon such as a hexagon or octagon or a greater number of rotational interlocking surface structures such as axially extending teeth on a gear and complementary axially extending grooves. At least 8 or 10 and depending upon construction materials at least 15 or 20 or more teeth and complementary grooves may be provided to increase the number of rotational alignments which will allow the resistance element to be mounted on the corresponding post.

The second aperture 134 is larger than the first aperture 130, and additionally comprises a keyed periphery so that it rotationally engages with a complementary second projection 136 carried by the resistance element 68.

The resistance element 68 is configured to provide resistance to relative motion of first projection 132 with respect to second projection 136. In this manner, the first lever 62 engages first projection 132 and second lever 64 engages second projection 136 so that rotation of first lever 62 with respect to second lever 64 about the axis of rotation is subject to the resistance provided by resistance element 68.

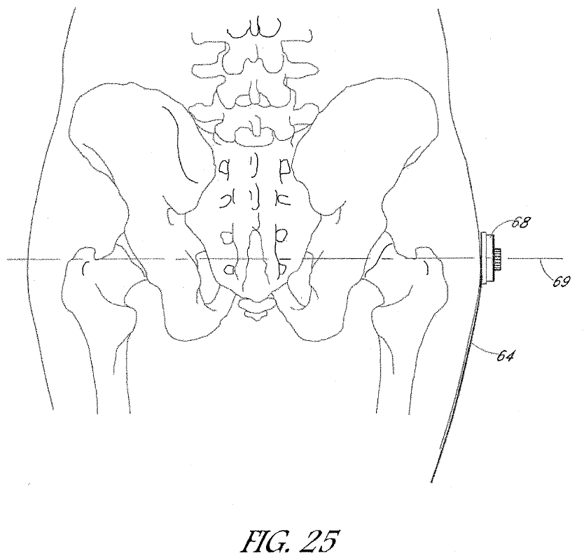

FIG. 22 illustrates an inverse configuration, where the garment carries post 74, attached to first lever 62. The second lever 64 is provided with a keyed ring 140 having an interior passage 138 for receiving post 74. Post 74 is provided with a keyed surface, and the cross-sectional dimension of passage 138 is sufficiently large that post 74 can rotate freely therein. Keyed ring 140 has a keyed exterior surface.

Post 74 extends through and beyond keyed ring 140 and is received within a first cavity 142 on the resistance element 68 and is rotationally locked therein. Keyed ring 140 is received within a complementary second cavity 144 and is rotationally locked therein. In one implementation of the invention, illustrated in FIG. 23, the keyed second cavity 144 is rotationally connected to the housing of the resistance element 68. Keyed post 74 is rotationally linked to an interior component of the resistance element 68 which rotates relative to the housing subject to the resistance provided by the resistance element.

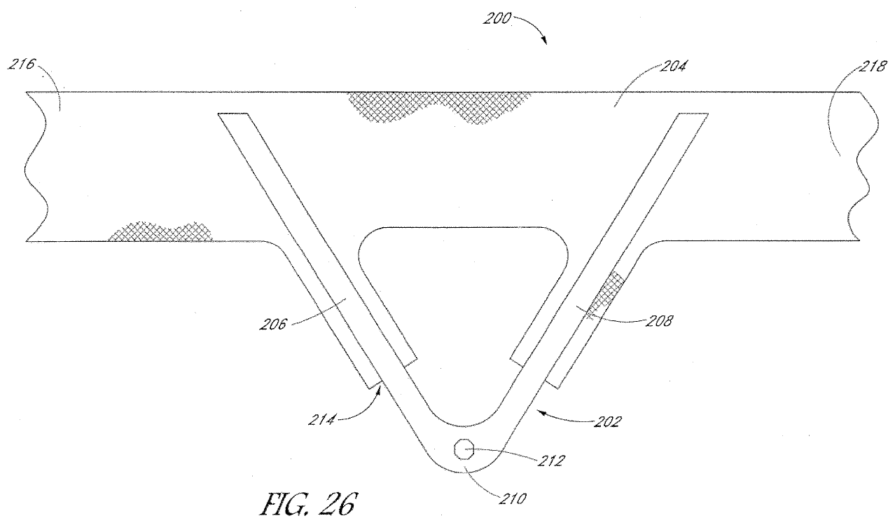

FIG. 24 illustrates a plan view of the first and second levers with keyed ring 140 fully seated on post 74, and ready for attachment of the resistance element 68.

Referring to FIG. 26, there is illustrated an alternative superior attachment assembly 200. The attachment assembly 200 comprises a lever 202 in the form of a “V”, having at least a first strut 206 and at least a second strut 208. First strut 206 and second strut 208 are provided with a force transfer layer 204 as has been discussed.

First strut 206 and second strut 208 are joined at an apex 210, which is concave in an upward direction in the as worn orientation. Apex 210 and force transfer layer 204 are configured to place the apex 210 approximately in alignment with the axis of rotation of the wearer's hip or other joint. Apex 210 is provided with a connector 212, which may include an aperture or post as has been discussed.

Each of first strut 206 and second strut 208 have a length within the range of from about 3 inches to about 8 inches, depending upon garment design. Each strut may have a width within the range of about 0.25 inches and about 2 inches, typically between about 0.5 inches and 1.5 inches, depending upon garment design, construction material and the intended resistance rating. Three or four or more struts may be connected to apex 210, depending upon desired performance.

Force transfer layer 204 on a first side of the wearer may have extensions 216 and 218 which extend in a circumferential direction around the waist of the wearer. Extensions 216 and 218 may be integral with or connect with the extensions on the superior attachment assembly 200 on a second side of the wearer.

The force transfer layer 204 may extend inferiorly along the length of the first strut 206 and second strut 208 to a transition 214. Above the transition 214, the lever 202 is securely attached to the underlying garment such as by way of the force transfer layer 204. Below transition 214, the lever 202 is unattached to the underlying garment, so that the apex 210 can float with respect to the underlying garment.

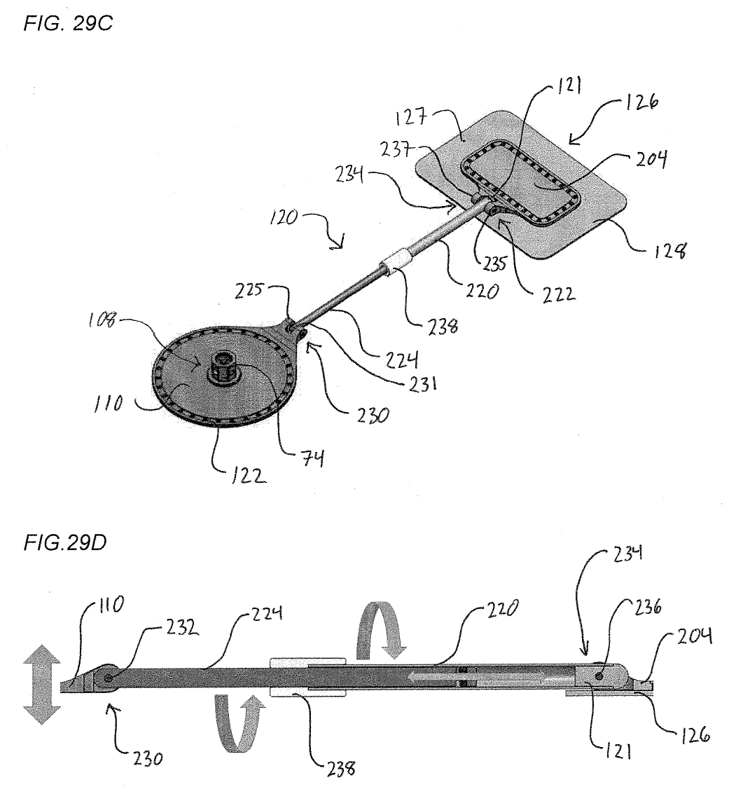

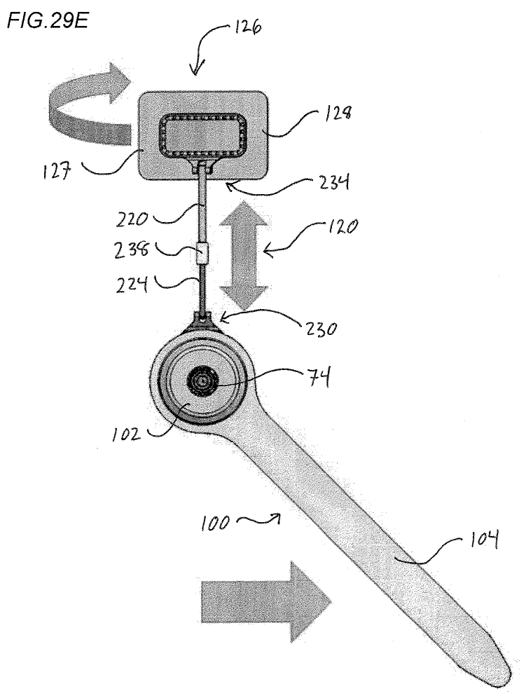

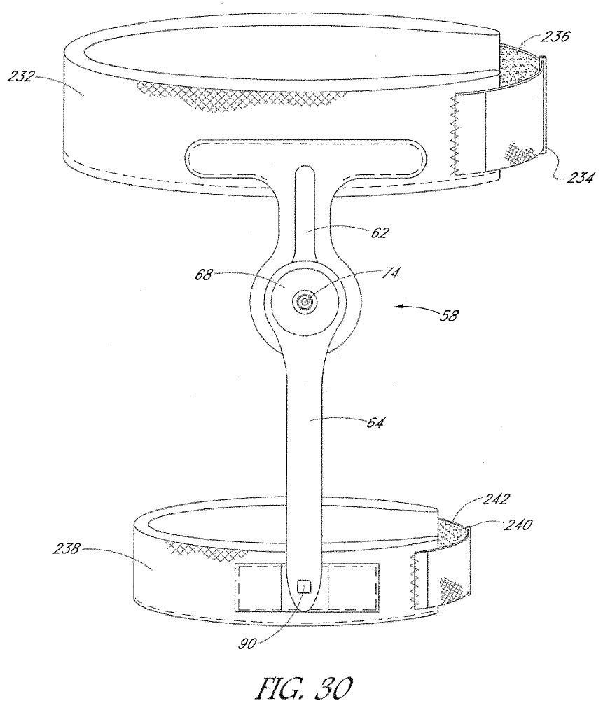

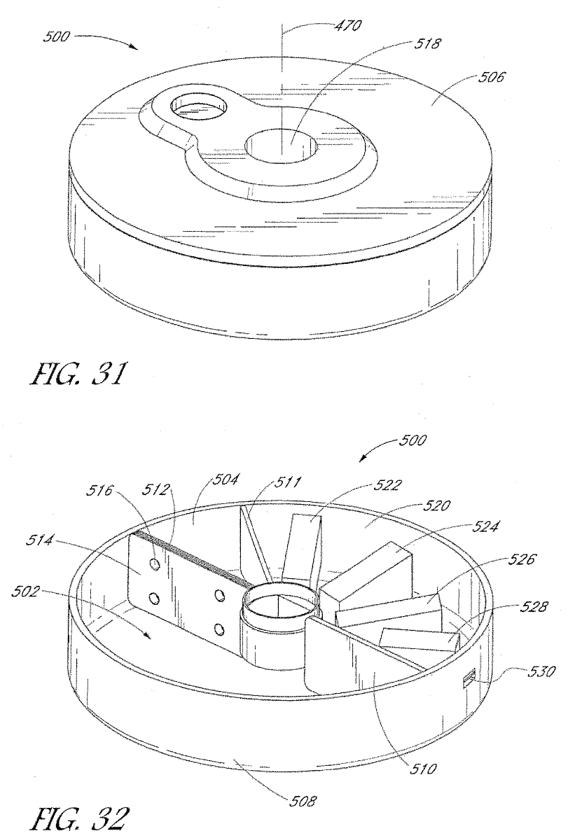

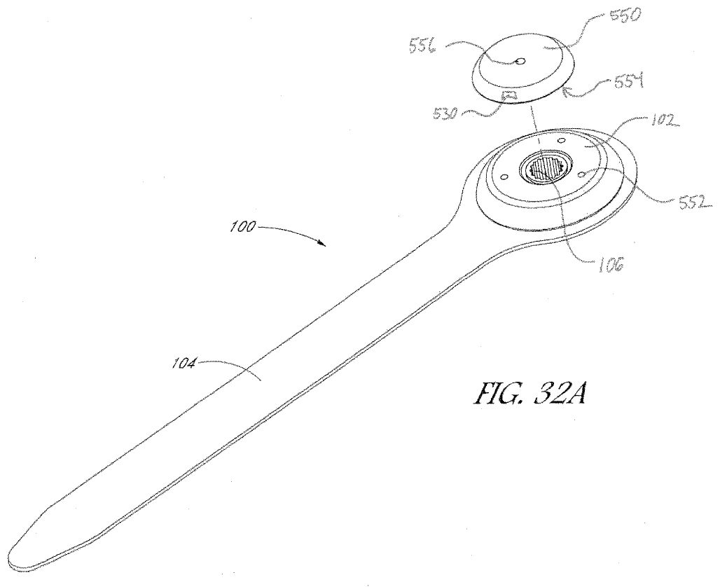

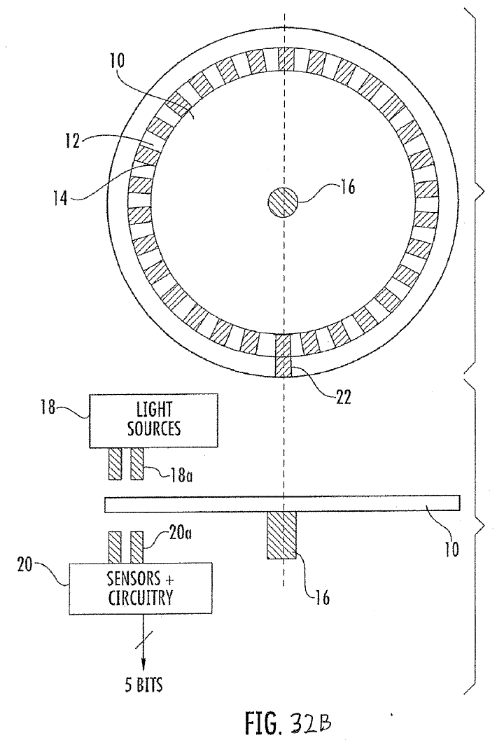

A superior attachment assembly 200 having multi axial adjustability is illustrated in FIGS. 27 and 29C. A tubular support 220 is securely bonded 222 to force transfer layer 204. Tubular support 220 is configured to axially slidably receive a rod 224 telescopically therein. The orientation of the sleeve and rod may be reversed as will be apparent to those of skill in the art. Rod 224 carries a connector such as an aperture or post 74, for engaging any of the resistance units describe elsewhere herein. The rod 224 may optionally also carry a docking plate from which the post extends. As illustrated in FIG. 29, a resistance unit 102 assembly may be mounted on the post 74.

In an implementation illustrated in FIG. 29A, at least the tube 220 and optionally the rod 224 have a circular cross-section. In this implementation, the rod 224 can rotate within the tube 220, allowing the resistance unit 102 to tilt from side to side about a vertical axis. This allows the resistance unit 102 to accommodate hip swivel movement of the wearer. If side to side adjustability is not desired, the tubular support 220 and corresponding rod 224 may be configured in a non-circular cross-section such as rectangular as illustrated in FIG. 29 B.

If the rod 224 remains axially slidably carried within tubular support 220, the post 74 is permitted to float up or down in the vertical relative to the force transfer layer 204 and or tubular support 220. This adjustability along a vertical axis allows the resistance unit 102 to float, and adapt to minor movements of the wearer and/or initial misalignment between the rotational axis of the resistance unit 102 and the rotational axis of the underlying joint. The range of float may be limited such as by providing opposing interference surfaces on the rod and sleeve, spaced apart by the desired range of float.

Single or double or more axes of adjustability may be provided in any of the embodiments disclosed herein. For example, the apex 210 of lever 202 illustrated in FIG. 26 may be provided with a vertically extending guide such as a tube, for axially and/or rotatably receiving a rod 224 carrying a connector such as a post 74. The post 74 may be directly coupled to the rod 224, with or without a docking plate as has been discussed elsewhere herein.

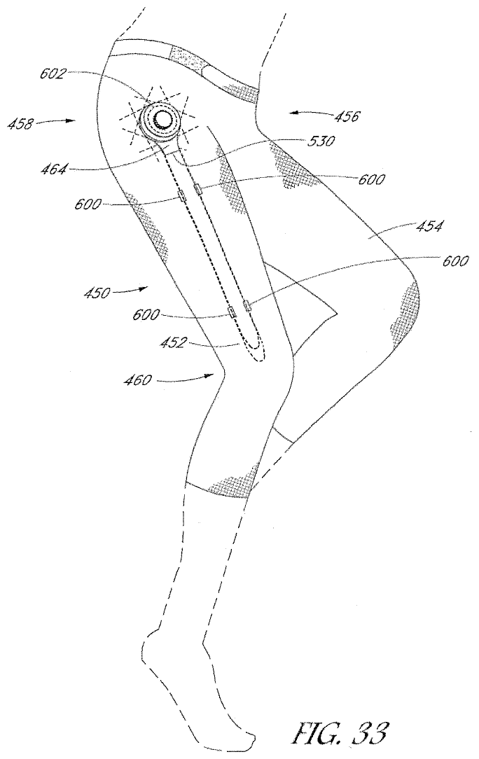

FIGS. 29C-E illustrate features of a multi axial adjustable docking assembly, that may be mounted on the resistance harness of FIG. 30 or any of the other compression garments or braces disclosed herein, in order to accommodate the complex movement at the hip relative to the waist. The connector 108 such as post 74 is connected to the superior lever 120 by at least one movable joint 230. In the illustrated embodiment, movable joint 230 connects the superior lever 120 such as rod 224 to the post 74, by way of docking platform 110. Movable joint 230 permits movement between the docking platform 110 and the superior lever 120 in at least one direction, such as medial lateral as illustrated. For this purpose, a pivot such as a hinge is formed by at least one projection 231 from the docking platform 110 overlapping at least one projection 231 of the superior lever 120, connected by and rotatable about a pin 232. Movable joint 230 may additionally be configured permit rotation of the docking platform and or post 74 about a longitudinal axis of the superior lever 120 (not illustrated).

The superior end of the superior lever 120 is preferably movably connected to the superior force transfer layer 204, which may be a portion of a garment, belt, or intermediate layer as has been discussed. In the illustrated embodiment, force transfer layer 204 is provided with at least one projection 235, and as illustrated two projections 235, 237, which define a space there between for receiving an extension 121 of the superior lever 120. An aperture is provided, for receiving a pin 236 similar as described in connection with movable joint 230. Movable joint 234 enables the force transfer layer 204 to flex in the medial lateral direction relative to superior lever 120.

The docking assembly may additionally enable rotation of the docking platform 110 with respect to the longitudinal axis of superior lever 120 as well as axial extension and retraction to accommodate changing distance between the force transfer layer 204 and the connector 108 under normal use conditions. This may be accomplished by allowing rod 224 to axially reciprocally move with respect to tubular support 220. For this purpose, the rod 224 is telescopically and concentrically slidably carried by tube 220. An annular collar 238 may be provided, at the inferior end of the tubular support 220, to provide controllable moving friction between the components, and exclude particulate, moisture or other debris from entering the tubular support 220.

In the as worn orientation, the force transfer layer 204 is securely held against the waist or other portion of the body above the axis of rotation of the hip. The docking platform 110 may be moved in an inferior and superior direction, rotated about the longitudinal axis of the superior lever 120, moved in a lateral medial direction, while maintaining a parallel plane to the force transfer layer 204, or be inclined such as about movable joint 230 or movable joint 234, in a medial or lateral direction to accommodate fit and movement at the hip.

FIG. 29E illustrates the assembly of FIGS. 29C and D, with an embodiment of a resistance unit and or electronics module mounted on a femoral lever as has been described elsewhere herein.

Referring to FIG. 30, there is illustrated a training harness in accordance with the present invention. The training harness may be configured for rapid attachment to the outside of a pair of pants or other athletic gear, or beneath clothing such as street clothing, or may represent a template for a subassembly to be integrated into a garment, and may be provided with any of the resistance elements and/or biometric features (e.g., power or other stride biomechanics measurement) disclosed elsewhere herein.

The harness 230 comprises a waistband 232, for removable attachment around the waist of the wearer. Waistband 232 may comprise a strap having foam padding. Waistband 232 is provided with an attachment strap 236 such as a Velcro strap attached to the waistband 232. An attachment structure such as a belt loop (buckle) 234 may be provided, for attachment using the Velcro strap. This construction enables a single device to be appropriately sized for any of a wide variety of wearers.

The harness 230 additionally comprises attachment structures for receiving a resistance unit 58, which is preferably bilaterally symmetrical (left and right resistance units). Any reference to a resistance unit herein is understood to also optionally include any of the power measurement or other sensors or electronics disclosed elsewhere herein, depending upon the intended product performance. The resistance unit 58 in general includes a connector for receiving a resistance element 68, along with a first superior lever 62 and a second inferior lever 64 as has been discussed. The resistance unit 58 may be removable and replaceable.

An inferior connector 90 connects the second lever 64 to a leg band 238. Leg band 238 is a flexible, padded band such as a compression sleeve configured to wrap around and secure to the leg of the wearer. For this purpose, an attachment such as buckle loop 240 may be provided to cooperate with a flexible strap 242 such as an elastic strap with Velcro attachment. The strap may be pulled through the belt loop 240 and secured to itself, to wrap the leg band 238 firmly around the leg of the wearer. One or two or three or more leg bands 238 maybe provided, for each leg, depending upon the intended load to be applied.

The harness 230 may be constructed of flexible, breathable lightweight materials which have relatively low stretch compared to some of the compression garments disclosed elsewhere herein. The levers 62, 64 may comprise strong, lightweight materials such as carbon fiber, metals such as titanium or aluminum or polymers known in the art. As such, the harness 230 may support resistance units having a much higher resistance to rotation, such as at least about 15 or 20 inch pounds, at least about 30 or 40 or 50 or more inch pounds of torque. The structural integrity of the levers and other harness components can be significantly reduced in an embodiment that collects data (e.g., rotary encoder) without imposing resistance to motion. As with other embodiments disclosed herein, the harness 230 is preferably bilaterally symmetrical although only a single side has been shown to simplify the drawing. The power modules and other biometric sensors and processors disclosed elsewhere herein may each be integrated into the harness 230 as will be appreciated by those of skill in the art.

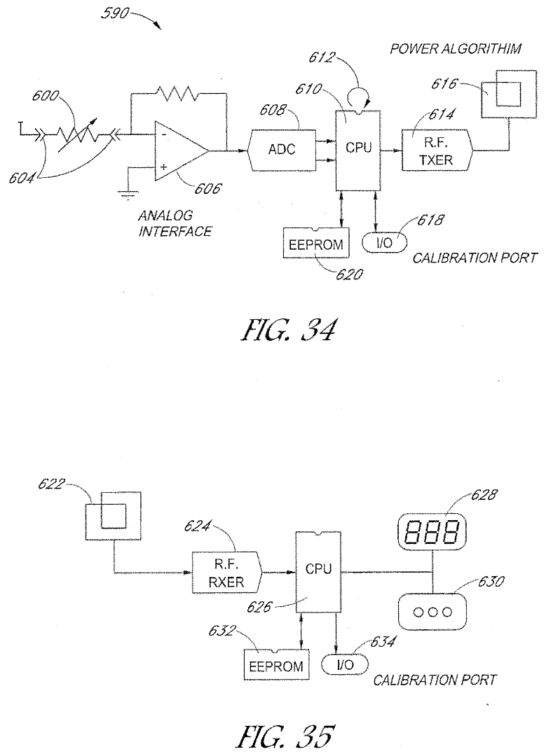

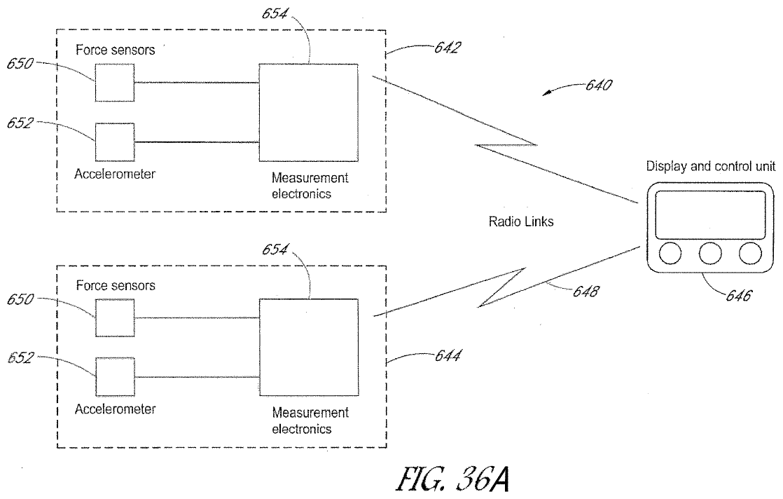

Referring now to FIGS. 31-32, a rotary damper resistance element is illustrated. Any of a variety of alternative specific damper constructions may be utilized as will be apparent to those of skill in the art. Linear dampers may also be used, along with associated lever arms, or mounted in line in a pulley system. The apparatus includes a housing 500 defining a housing interior 502 for containing damper fluid (not shown) of any conventional nature, and optimally also electronic components in an implementation in which the biomechanics and or electronics unit is integrated into the resistance unit. The housing interior has a substantially circular cross section and is formed by a toroidal or cylindrical (illustrated) inner housing surface 504 disposed about and spaced from a central axis 470. The illustrated housing 500 includes two adjoining housing members 506, 508, each housing member defining a portion of the housing interior.

At least one vane or piston 514 having an outer peripheral piston surface at which is located an outer seal 512 is in substantially fluid-tight, slidable engagement with the inner housing surface, spaced from axis 470 and disposed along a common plane with the axis 470. The housing 500 and the piston 514 are relatively rotatably moveable about the axis, as will be described in greater detail below.

A first fluid barrier 510 and a second fluid barrier 511 which may each be in the form of a plate are immovably attached to the housing and positioned in the housing interior.

The vane 514 defines multiple flow control orifices or passageways 516 which permit restricted passage of damper fluid therethrough responsive to relative rotational movement of the vane 514 throughout an angular range between the first fixed barrier 510 and second fixed barrier 511 to dampen forces applied to the apparatus causing the relative rotational movement.

A shaft or aperture 518 extends through the housing interior along axis 470 and is exposed on at least one opposed side of the housing, for connection as has been discussed.

Piston 514 is secured with respect to shaft or a sidewall of aperture 518 such that relative rotational movement between the housing and the boundaries of the aperture 518 causes the piston 514 to rotate through an arc about axis 470. This will cause damper fluid in the housing interior to pass through flow control passageways 516 and thus resist the relative rotational movement.

In the illustrated embodiment, the barriers 510 and 511 define a first portion 504 of the housing interior 502 for containing viscous fluid, and enabling piston 514 to rotate throughout an angular range of motion. The hip normally rotates in the anterior posterior plane throughout a range which varies from individual to individual and based upon speed of travel, but is generally from about 35° for short walking strides to a maximum of no more than about 120° for most wearers. The knee, elbow and other motion segments also have a limited range of motion. Thus a full 360° range of motion at the resistance unit may be provided but is not normally necessary. The barriers 510 and 511 in the illustrated embodiment thus also define an electronics component chamber 520 which is isolated from the damper chamber 504. Electronics component chamber 520 may include any of a variety of electronic components described elsewhere herein, depending upon the functionality of the device. For example, a power supply 522 such as a battery may be provided. Also illustrated is a central processing unit 524, a transmitter or transceiver 528 and potentially one or more sensors 526.

The electronics component chamber 520 may alternatively or additionally be carried in a separate removable, interchangeable electronically enabled module 550 as illustrated in FIG. 32A. The electronics module 550 comprises a housing having at least one chamber or space therein for containing any one or more of the electronic components or systems disclosed elsewhere herein. The housing has a lower docking surface 554 having at least a first connector (not illustrated) configured to releasably connect to a second, complementary connector 552 on a resistance unit 100 or resistance element 102. Any of a variety of mechanical interference fit structures may be used for snap fit, threaded fit or other releasable engagement. One or two or three or four or more complementary pairs of connectors may be utilized. Magnetic attachment may also be used, with magnets carried by the resistance element positioned to align with complementary magnets of opposite polarity in the electronics module 550. ElectroPermanent Magnets or EPM's may be desirable, since the external magnetic field can be turned on and off by applying a current pulse, but no current is required to maintain the magnetic field once the EPM has been activated.

The electronics module 550 is also provided with a rotatable shaft or other rotation sensing or transferring element 556, to couple to the rotatable aperture or shaft of the resistance element. One or more electrical connections may also be provided on the docking surface 554, for placing the electronics module into electrical connection with electrically operated components within the resistance element 102. For example a multiple pogo pin connector on one docking surface can be brought into alignment with a complementary multi conductor pogo connector on the other complementary docking surface. Inductive communication may be desirable since it may have better durability in a damp environment. Electrical communication between the electronics module and the resistance unit may be desirable if some electronics such as certain sensors are preferably located within the resistance module or elsewhere on the garment.

An electronics module 550 may be multipurpose, and include electronics to enable any combination of functions and measure any of the biometrics described elsewhere herein. Alternatively, application specific modules may be produced to help reduce cost and tailor functionality to a particular wearer's needs. For example, a module may be configured to report any one or combination of incremental power, stride rate, stride length, or derived metrics such as power to heart rate ratio; power to weight ratio; efficiency factor, right-left balance or imbalance for any relevant parameter, vertical to horizontal power ratio or more depending upon the intended use.

The electronics module 550 may be configured solely as a data capture biomechanics unit, to be downloaded following the exercise period. It may alternatively be configured as both a data capture and transmit device, such as to transmit raw or processed data to a remote receiver, with or without any direct proprioceptic feedback to the wearer. The remote receiver may be a smart phone, tablet computer, wrist watch or other device capable of receiving and displaying the data, for use by the wearer, a coach, medical personnel, or anyone who has a desire to see performance metrics. Multiple players or athletes on a team may simultaneously transmit performance data to the coach, who can monitor power output, heart rate and other metrics disclosed herein of the team members side by side as they go through similar activities, for various comparative evaluation purposes.

Power supply 522 may comprise a battery pack, which may be carried within the housing 500 in a permanent or detachable manner. Any of the electronics components disclosed herein may be carried in the housing 500, module 550 or adjacent structures such as a separate electronics module carried by the garment or by the wearer separate from the garment. The battery pack may represent a one-time-use, disposable battery or may represent a rechargeable battery pack (e.g., Lithium-Ion, Nickel Metal Hydride, or the like) to be recharged for use via a charging port (e.g., a micro USB connector 530) provided with a water resistant cap or plug. Charging may alternatively be accomplished via a wireless charging technology such as inductive charging via an induction coil carried by or within the housing. The battery pack (rechargeable or otherwise) may be configured to be replaceable (e.g., by the user) in the event the battery fails or to swap out a battery with low charge or no charge, with a freshly charged battery, for example. Battery pack may be configured to accept batteries with different amp-hour capacities to provide sufficient duration of operation of the garment and its associated electronics, such as 1500 mAh, 3000 mAh, etc. Power supply 522 may alternatively comprise an on board generator, such as a rotational generator positioned at the hip or knee to take advantage of reciprocating joint rotation. Other energy scavenging sources can take advantage of body temperature, respiration, stride (e.g., foot strike) temperature change representing calories burned as a result of movement at the hip, which elevates the temperature of the damper, or others as is understood in the art.

Communication module 528 to permit electronics on the resistance unit and/or carried elsewhere on the garment to communicate (e.g., wireless data) with one or more of external, remote devices such as a smart personal communication device (e.g., a smart phone, tablet, or pad), remote feedback device, on board feedback device such as a vibrator, compression pad or ring, electrical current or other feedback effector, or any of a variety of tracker systems such as those produced by Fitbit, Jawbone, Nike's Fuelband or Under Armour's Healthbox connected ecosystem. Typically, wireless communication among components of the wearable fitness ecosystem may employ any suitable air interface, including for example Bluetooth™ (in its various implementations, including low power Bluetooth), ANT™, ANT+, WiFi™, WiMAX™, 802.11(x), infrared, cellular technology (such as for example GSM™, CDMA™, 2G™, 3G™, 4G™, 5G™, LTE™, GPRS™), etc. The selection of the appropriate air interface for communication depends on the air interface availability in the devices and/or at the location, cost, convenience, battery life and/or other factors.

The sensor module 526 can include any of a variety of sensors described elsewhere herein, depending upon the desired functionality. For example, temperature sensors may be provided both to enable correction of other sensor data or electronics (e.g. strain gauges) due to thermal drift as the resistance unit rises in temperature, as well as to provide a metric of calories burned. Sensors for enabling the determination of force, power, stride length, stride velocity, stride rate, acceleration among others may be conveniently placed on or within the resistance unit. For example, at least one or two or four or more accelerometers, rotary encoders or other sensors disclosed elsewhere herein may be placed throughout the resistance unit, femoral lever or garment (e.g., left and right arm; left and right leg) and/or otherwise carried by the wearer's body (i.e., attached via any suitable manner to shoes, wrist bands, etc.) to collect multiple data points. Each of the additional accelerometers or other sensors may be connected wirelessly or via electrical conductors back to the controller 524 and/or communication module 528. A suitable 3-axis accelerometer may be a model ADXL377 available from Analog Devices, Inc. of Norwood, Mass. or any equivalent. Likewise, a suitable 3-axis gyroscope may be a model ADXRS652 available from Analog Devices, Inc. of Norwood, Mass. or any equivalent.

Raw data may be sent from a 3-axis accelerometer and/or a 3-axis gyroscope and/or a rotary encoder, for example to the controller 524 which can record rotation, acceleration, 3-axis gyroscope position in terms of x, y, and z coordinates. The controller 524 may obtain position point recordings multiple (e.g., 500) times a second and is configured to automatically write the data points to memory along with transmitting the data over the communication interface to sensor data interpretation software which may be resident on a remote computing device (e.g., laptop, cell phone, etc.). Additional details of wearable gyroscope and accelerometer systems may be found in US patent publication 2014/03133049 to Doherty, the entirety of which is hereby incorporated by reference herein. Strain gauges, piezoelectric and proximity sensors may also be mounted on the resistance unit depending upon a variety of manufacturing choices and intended functionality.

The controller module 524 may also include processing electronics for performing some or all required signal processing on the sensed signals. In one or more embodiments, such signal processing (e.g., amplifying or filtering) may be performed locally in one or more of the sensors at the controller 524, or both, for example. Controller 524 may also include signal processing for performing data analysis and feedback data generation. In one or more embodiments, such data analysis and feedback data generation may be performed at one or more of controller 524, local remote device such as a fitness tracker or smart phone or the Internet. Signal processing for performing data analysis and feedback data generation may occur solely in the garment and its associated electronic circuitry, external to garment, or both where some portion of the processing is done in the garment and other portions are done external to the garment using processors and resources of external devices and/or systems.

Controller 524 may include one or more processors, multi-core processors, one or more digital signal processors (DSP), one or more micro-processors, one or more micro-controllers, one or more application specific integrated circuits (ASIC), one or more field programmable gate arrays (FPGA), one or more analog-to-digital converters (ADC), one or more digital-to-analog converters (DAC), a system on chip (SoC), one or more operational amplifiers, custom logic, programmable logic, analog circuitry, mixed analog and digital circuitry, or the like, just to name a few. Alternatively, raw or partially (incompletely) processed sensor data can be transmitted to an electronics module carried elsewhere, such as on a belt, or off board to a cellphone or other smart local remote device where data manipulation is accomplished. This shifts the weight, power consumption and expense of computational components off board of the garment.

Analysis performed either on board the controller 524 or off board may include, in one or more embodiments, comparing an exertion level (e.g. power expenditure, or power/heart rate, power/weight, vertical/horizontal power type ratios) with the reference exertion level as is discussed elsewhere herein. Other sensor data such as rotary encoders, bend-angle sensor data or accelerometer sensor data may be used to compare parameters such as acceleration, velocity, other motion or position to the reference data.

Analysis may also include, alternatively or additionally updating a user profile and comparing against profiles of one or more other users. In one embodiment, user profile data may include a history of workout sessions including overall exertion (e.g., total watts, watts/kg etc.). In another embodiment, profile data may include goals set by the user and additionally or alternatively challenges from other users (e.g., to motivate the user). For example, the challenges may come from other persons or users who may be associated with a social network (e.g., Facebook®, Twitter®), professional network (e.g., LinkedIn®), training partner, training team, coach or the like. Through social and/or professional networking of user profiles including historical workout data, motivation is increased by the competitive environment created. Additionally, challenges or goals may be proposed by the system (e.g., controller 524 and/or other system in communication with controller 524). A combination of progressive challenges (e.g., a series of challenges, each with higher goals to be achieved) may lead the user to higher and higher levels as in a gaming scenario where gameificaiton of the challenges may comprise the user taking on progressive challenges against goals set by the user, the system, others, or by other competitors in the game, for example.

As will be apparent to those of skill in the art in view of the disclosure herein, certain sensors are preferably mounted elsewhere on the garment but other sensors may be or preferably are mounted at or near the axis of rotation on the damper or damper housing. These may include force sensors, angular displacement sensors, accelerometers, proximity sensors, (potentially depending upon the manner in which data is obtained for the calculation of power) and temperature sensors, such as to directly measure caloric burn accomplished by the resistance unit. An external electrical connector 530 such as a mini USB port may also be provided on the housing, for electrical connection to an external device such as to charge the battery 522, program the CPU, and or download data which has been obtained during an exercise period or other data collection period. The CPU module may contain memory, and or a separate memory module may be provided depending upon the intended length of the data collection period and or the complexity (i.e., data rate) of the data being recorded.

Referring to FIG. 33, there is illustrated a training garment 450 having a right leg 452 and a left leg 454. The training garment preferably comprises at least one stretch panel, for providing a snug fit and optional compression. The panel may exhibit stretch in at least a circumferential direction around the leg and waist. Stretch panel may comprise any of a variety of fabrics disclosed elsewhere herein.

Resistance garments in accordance with the present invention can be configured as independent biometric sensing and feedback devices, or can be configured to communicate and/or cooperate with external electronic systems and devices, such as cell phones, the internet, local area networked devices and particularly activity tracking devices such as those produced by Fitbit, Inc., San Francisco, Calif. (see, for example, U.S. patent application Ser. No. 13/156,304, filed on Jun. 8, 2011, entitled “Portable Monitoring Devices and Methods of Operating Same” published as U.S. 2012/0083715 which is incorporated herein by reference in its entirety).

Biometric and/or ambient condition, spatial location, motion or other sensors and processing circuitry may be carried by the resistance unit (e.g., within the resistance element or within a detachable module attached to the electronics module and/or the resistance unit or resistance element), integrated into the garment or other support associated with the resistance element, or may be separately worn by the wearer such as when the garment is configured to pair with a wearable activity tracker such as any of a variety of Fitbit models. One or more sensors carried by the electronics module, resistance unit, garment or the wearer of the garment can include, for example, electromyography (EMG), electrocardiograph (ECG), respiration, galvanic skin response (GSR), temperature, acceleration, bend angle, pressure, force, torque, GPS, accelerometer (single or multi axis), respiration, perspiration, bioimpedence, gyroscopes, various rate measurements such as stride rate, flex rate, pulse (heart) rate, spatial orientation, deviation or position, oxygen saturation, blood glucose, or others described elsewhere herein. Sensors may also be provided to detect, measure and/or sense data which is representative of hydration, height, weight, sun exposure, blood pressure and/or arterial stiffness. See, for example, U.S. patent application Ser. No. 14/476,128, filed on Sep. 3, 2014, entitled “Biometric Monitoring Device Having a Body Weight Sensor and Methods of Operating Same” published as U.S. 2014/0379275, which is incorporated herein by reference in its entirety. The use of multiple sensors for the same parameter or multiple sensors for multiple parameters may provide a level of insight that is not available by measuring only a single metric such as heart rate (HR) or motion based on accelerometers or other types of motion sensors (e.g., a gyroscope). Sensors may be incorporated in a permanent manner into the fabric of the form-fitting interactive garment itself or in a detachable manner such as with zippers, snap fit connectors, clasps, hook and loop (Velcro) or other releasable connectors and/or in pockets or under or on top of flaps if desired, to allow removal and/or repositioning of the sensors.

Biometric or other data parameters and/or data derived from biometric or other parameters can be displayed and/or stored for subsequent display in a form that indicates an incremental effect of the resistance provided by a resistance element in accordance with the present invention. For example, a wearer might walk for 1,000 actual steps. If those steps were taken while wearing a resistance garment as disclosed herein, a ‘steps equivalent’ may be calculated and displayed indicating the equivalent number of steps that would have been required to have been taken to have burned an equivalent amount of calories or perform an equivalent amount of work. So the 1,000 steps with a first resistance level rating might be an equivalent amount of work to 1,100 actual steps without the resistance unit. Thus the resistance garment produced an incremental 10% energy burn or effort over steps taken without the resistance elements. A second resistance level unit might enable 1,000 steps to be equivalent to 1200 steps without the resistance unit. Fixed resistance units can be provided at a variety of resistance levels, configured to produce an incremental burden of at least about 10%, 20% 30%, 50% 75% or more in excess of the burden incurred by the activity such as walking in the absence of the resistance unit. In configurations designed more for athletic training than toning, potentially incremental loads of at least about 100% or 150% or 200% or more over the unburdened baseline may be desirable. [01%] The incremental effect of the resistance units can be expressed in various other ways, such as incremental power (Watts) or incremental calories burned. So if 2,500 steps would normally burn 1100 calories for a particular wearer in the absence of a resistance garment, the same 2500 steps might burn at least about 10% or 20% or 30% or 50% or more incremental calories for the same 2500 steps while wearing a resistance garment. The incremental effect can alternatively be calculated as an effective slope equivalent. A baseline slope can be selected, such as horizontal. Walking along a substantially horizontal surface while wearing a resistance garment, depending upon the resistance level, might be the equivalent of walking uphill along a slope of plus at least about 4 degrees, at least about 10 degrees, at least about 15 degrees at least about 20 degrees or more.

Incremental elevation or change of respiration rate, pulse rate, blood gas such as CO2 or O2, temperature, blood glucose may be measured by a sensor or calculated, so that the wearer, care provider or friends connected via social media or other networking environment can see the physiological benefit provided by wearing the resistance units of the present invention.

Synchronization between the wearable resistance device and a wearable activity tracker can be accomplished either automatically (e.g. wirelessly) or manually. For example, in the example above of a resistance garment carrying a resistance unit which is rated to provide an incremental 20% calorie burn or resistance to walking, a code carried by the resistance unit corresponding to the level of resistance can be input into the activity tracker, and the activity tracker programmed to calculate the parameter equivalent accomplished by the wearer while utilizing that resistance element. So the activity tracker can reflect that the actual 1000 steps with the resistance unit was the equivalent of 1200 steps without the resistance unit.

More simply, the activity tracker can be programmed to receive an input of a factor corresponding to the resistance value of a particular resistance unit. The factor would cause the activity tracker to report the effective value (e.g., 115 steps) rather than or in addition to the actual value (e.g., 100 steps) for the parameter of interest.

Alternatively, the activity tracker may be caused to periodically or on-demand ping an interrogator signal. The resistance element or the garment carrying the resistance element may be provided with a RFID or other identification tag or circuit which can reflect a signal back to the activity tracker, indicating the resistance rating. The activity tracker can then calculate an equivalent value for a parameter of interest being displayed or available for display, indicating the incremental change relating to that parameter caused by the resistance element. In more complex systems, the resistance element, activity tracker and optionally sensors carried by the garment can be in communication using any of a variety of wired or wireless protocols such as ANT, ANT+, Bluetooth, WiFi, ZigBee or others known in the art.

Thus, an activity tracker configured to pair with the resistance garment of the present invention may be provided with an input, configured to receive a compensation factor which will enable conversion of a measured or calculated parameter into an equivalent, taking into account the effect of the resistance units on the measured parameter. The input may be configured for the user to manually input the compensation factor. Alternatively, the input may be configured to wirelessly receive the compensation factor from the resistance unit. The activity tracker may be configured to record and or display or output the equivalent value, and optionally also the actual value of the parameter of interest. For example, the activity tracker may be configured for receiving an input indicating that each actual step will require the wearer to exert 1.2 steps worth of effort. The activity tracker will therefore display 120 step equivalents for every one hundred actual steps taken by the wearer, while the corresponding resistance element is engaged.

For embodiments of the present invention utilizing a viscous damper, the resistance to movement will vary as a function of angular velocity. For any of the embodiments disclosed herein, and particularly for viscous damper embodiments, it may therefore be desirable to measure actual power rather than merely calculating a metric of work based upon the number of repetitions. Preferably, the level of exertion will be described in terms of wattage (intensity) and Joules of work (quantity) being done, from which calories burned can be determined and displayed or saved.

A variety of power sensors are known in the performance bicycle arts, which may be readily adapted for use in the present context. Typically, a power sensor such as a strain gauge will be positioned such that it captures force exerted by the wearer. Power sensors maybe positioned in a variety of locations on the garment, such as on the anterior side and or posterior side of the lower limit of the garment (knee or ankle), and/or carried by the resistance unit and its attachment structures. Torque or other angular sensors may be attached to the resistance unit, and/or the mounting station for receiving the resistance unit. All may be provided with wired or wireless communication back to a central processing unit carried by the garment, or to a remote device such as the activity tracker, cell phone, or other as has been described. Although power output by the wearer is perhaps most conveniently measured by utilizing the relative rotation of the femoral lever with respect to the hip, wireless power output sensors may be positioned elsewhere in the garment, and configured such as those disclosed in United States patent publication 2015/0057128 to Ishii, the disclosure of which is hereby incorporated by reference in its entirety herein.

Any of the configurations disclosed herein may additionally be configured to determine and display a metric of total or incremental power (e.g., in Watts) expended by the wearer, or incremental calories burned, as a result of movement against the resistance provided by the resistance unit. For example, referring to FIG. 33, at least one or two or more sensors 600 may be positioned in the force path between a first surface connected to the resistance element such as on the femoral lever arm, and a second surface mechanically connected to the wearer, such as an interior opposing force transmission surface within the sleeve. Split lever arms may also be provided with a sensor positioned to be under compression or shear between a first and second surfaces on corresponding first and second portions of the lever arm when the wearer moves against the resistance.

In one configuration, at least a first, anterior sensor is provided on an anteriorly facing surface carried by the lever arm. The first anterior sensor will be under compression as the wearer moves their leg rearward (in extension). At least a first posterior sensor is provided on a posteriorly facing surface carried by the lever arm. The first posterior sensor will be under compression as the wearer moves their leg forward (in flexion). Two or three or more sensors may be provided to measure force upon flexion or extension such as to improve accuracy of the reading. Preferably, sensors are bilaterally symmetrical so that comparative left-right data can be generated and displayed to reveal any bilateral asymmetries for any measured parameter.

Alternatively, force sensors 602 may be mechanically connected to the damper connector such as the aperture or shaft or otherwise configured to measure force at the point of rotation as in understood in the art. Signals from any or a combination of sensors 600 and 602 may be used to calculate a metric of power (e.g. force or proximity) expended by the wearer to move against resistance provided by the resistance element. One system having strain gauges embedded in the hub of a rotating construct for the purpose of measuring power is disclosed in U.S. Pat. No. 6,418,797 to Ambrosina et al., the disclosure of which is hereby incorporated in its entirety herein by reference. In another construction, the axel or post 474 is configured to undergo slight deformation in response to applied torque, and sensors are positioned to measure strain as that deformation occurs. Additional details may be found in U.S. Pat. No. 6,356,847 to Gerlitzki, the disclosure of which is hereby incorporated in its entirety herein by reference. Force or power data can alternatively be sent to the processing electronics from other sensors such as sensors carried by or mounted within the wearer's shoes.

As an alternative or in addition to sensors that directly measure force, a variety of position sensors can be incorporated into the detachable electronics module 550, resistance element 102 or other housing, along with or without the resistance mechanism. The position sensors are configured to detect angular orientation at the hip or other measured joint or motion segment and time, and its rate of change. Suitable sensors include a capacitive transducer, a capacitive displacement sensor, an eddy-current sensor, an ultrasonic sensor, a grating sensor, a Hall effect sensor, a magnetic sensor, an inductive non-contact position sensor, a linear variable differential transformer (LVDT), a differential transformer, a linear variable displacement transformer, a linear variable displacement transducer, a multi-axis displacement transducer, a photodiode array, a piezoelectric transducer, a potentiometer, any of a variety of rotary encoders, a string potentiometer, or a small CCD or CMOS video camera, depending upon the desired performance.

A variety of rotary encoders (e.g., capacitive, magnetic, optical, variable resistance, transmissive, reflective, etc.) may be utilized to capture angular position and time. For example, FIG. 32B schematically illustrates an optical incremental encoder that may be incorporated into the power module or biomechanics unit of the present invention. The rotary encoder comprises a disk 10 comprising at least one annular ring of alternating clear and opaque sections 12,14. The disk can be formed from glass, plastic or metal among other materials, and the clear (transmissive) and opaque sections 12,14 can be formed by etching, printing, embossing or any other suitable method. The disk 10 is fixed to a pin 16 which may be secured to the femoral lever, such that the pin and disk rotate in use relative to a housing. Referring to FIG. 32B, the disk 10 is interposed between one or more light sources 18 and one or more corresponding sensors 20 with associated circuitry, and positioned such that the light sources 18 and sensors 20 are arranged on either side of the circumferential edge comprising the clear and opaque portions 12,14. In a reflective configuration, the light sources and sensors may be positioned on the same side of the disc.

As the disk 10 rotates in response to movement across a joint such as the hip, the intensity of light incident on the sensors varies as the clear and opaque patterns 12,14 pass the under the light sources 18 in sequence. The measured intensity is amplified or fed into a comparator to produce a sign wave or digital square wave. The pulses of this output are then counted by the circuitry associated with the sensors 20 to give positional information as the intensity of the light varies.

In order to yield an absolute angular position, a known a reference point is provided. This can be in the form of a single opaque section 22 on an outer track of the disk 10 or by use of a separate mask component as is well known in the art. In one embodiment, a first light source and sensor pair is provided for detecting the varying intensity of the track comprising the alternating clear and opaque parts 12,14, and a second light source 18a and sensor 20a pair is provided for the detection of the reference point 22. Additional details may be found in U.S. Pat. No. 7,777,879, the entire contents of which are hereby incorporated by reference herein.

In a capacitive rotary encoder, a disc is provided with a pattern of (typically sinusoidal) metal lines and mounted to a drive shaft for rotation between a transmitter and a receiver. As the central disc rotates in response to a stride of the wearer, the capacitance between the transmitter and receiver changes, thus providing time and position information about the angular orientation of the disk relative to the housing.

The determination of a biometric parameter such as expended power can be accomplished on only one of the right side or left side of the wearer, such as at the right hip or hip plus knee but not the opposing side. The value can be doubled, under the assumption that the wearer's exertion will be bilaterally symmetrical. Preferably, the sensor system will be bilaterally symmetrical on both the right and left side of the wearer, to allow the wearer to see separate values for right and left side performance or an indication of deviation and evaluate any asymmetries in power output or other parameter and adjust training accordingly.

Based at least in part on torque and angular velocity of the leg of the wearer, instantaneous, average, peak, maximum, and/or minimum, horizontal and vertical power exerted by the wearer can be determined and displayed or utilized for further data processing operations such as to generate ratios as is discussed elsewhere herein. Total energy or power exerted by the wearer can be approximated based at least in part on one or more of the wearer's weight, stride rate, stride length, height, running speed, or any combination of these. These values can be provided to the wearer to provide feedback regarding power exertion during exercise.

Resistive torque (e.g., a resistance to movement of the thigh of the wearer) provided by RVD type resistance units is related to the angular velocity and/or angular acceleration at the hip. One or more sensors can be provided to measure the angular velocity. These measurements can be used to determine the resistive torque applied by the resistance unit (e.g., the torque that the wearer needs to overcome to move their thigh). For example, the resistance unit can have a look-up table or other function that maps angular velocity to resistance or resistive torque.

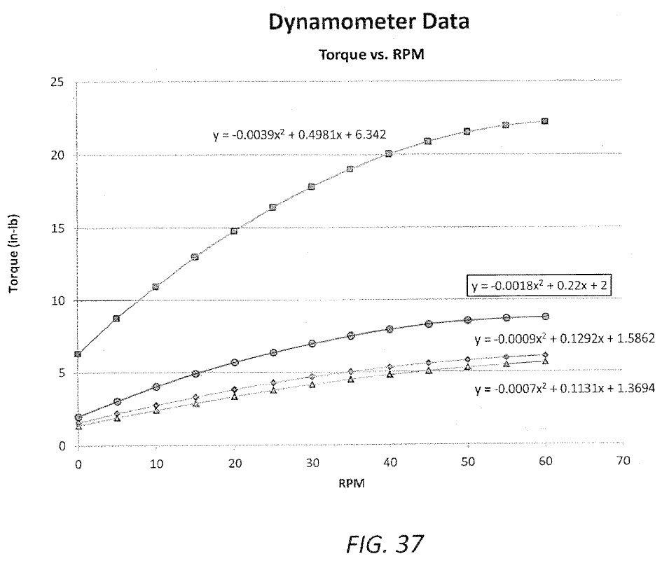

For example, FIG. 37 illustrates the torque characteristics for three resistance elements in accordance with the present invention, plotted against RPM (which can be readily converted to degrees per second, a unit used elsewhere herein). So at any point throughout the stride, the angular velocity can be measured and the torque applied by the resistance unit at that velocity can be determined from the torque v RPM data for that resistance unit. The torque data can be built into software carried by the electronics module, or maintained off board such as on the smart phone, activity tracker or other remote device.

As described herein, strain gauges or other measurement devices can be provided that measure force and/or torque applied by the wearer on the resistance unit. If the torque applied by the wearer exceeds the resistive torque, then the wearer's thigh can move. The difference between the applied torque (torque applied by the wearer) and the resistive torque (torque applied by the resistance unit) is the net torque. This net torque can be used at least in part to determine the mechanical power or energy being provided by the wearer.

In some embodiments, the net torque can be used to determine, measure, or estimate the energy or power exerted by the wearer. The instantaneous power can be determined as the product of the net torque and the instantaneous angular velocity of the wearer's thigh (e.g., P=τ*ω, where τ is the net torque and ω is the instantaneous angular velocity of the thigh). The peak or maximum power can be determined by sampling the instantaneous power over time (e.g., over at least about 1, 2, 5, 10, 20, 50, etc., strides) and determining a maximum power over that time. Similarly, the peak or maximum power can be determined by sampling the instantaneous power over a number of strides, determining a maximum power within each stride, and determining an average or median of the maximum power over the number of strides. The average (median) power can be determined by averaging (determining the median of) measurements of the instantaneous power. Similar processes can be employed to determine other statistical properties of the power. Furthermore, similar calculations and procedures can be followed for determinations of energy or mechanical work exerted by the wearer.

If the angular velocity is not measured or otherwise determined, the instantaneous angular velocity can be estimated in a variety of ways. Some methods for determining instantaneous angular velocity include determining a stride rate and then calculating an estimated instantaneous angular velocity based at least in part on statistical models associating stride rate with thigh position. In certain implementations, the stride rate can be estimated based on a plurality of measurements of torque. The measurements of the torque can be used to estimate the stride rate of the wearer by identifying cyclical patterns within the torque measurements to determine the beginning and endings of strides of the wearer. In various implementations, sensors can be used to determine the stride rate of the wearer (e.g., sensors such as accelerometers, gyroscopes, pressure sensors, or the like can be used). In some implementations, the stride rate can be entered or provided by another system or by the wearer.

As an alternative to direct measurement, the stride rate can be estimated based on predicted or typical stride rates of runners. For example, a typical recreational runner may have a stride rate between about 150 and about 170 steps per minute. As another example, competitive runners typically have a stride rate between about 180 and about 200 steps per minute. As another example, sprinters can have a stride rate that exceeds about 200 steps per minute. The typical stride rate for a person walking can range between about 100 steps per minute to about 150 steps per minute.

With the stride rate determined or estimated, the instantaneous angular velocity can be determined based at least in part on a statistical model of the relationship between a phase of the stride and thigh position. For example, the thigh position at various relative times within a stride is statistically similar across adults. This can depend at least in part on the speed of the wearer's gait (e.g., walking, running, sprinting, etc.). A walking adult typically has a thigh angle that varies about 50 degrees (e.g., between about 45 and about 55 degrees, or between about 40 degrees and about 60 degrees) over a single stride. A running or jogging adult typically has a thigh angle that various about 55 degrees (e.g., between about 50 and about 60 degrees, or between about 45 degrees and about 65 degrees) over a single stride. A sprinting adult typically has a thigh angle that various about 60 degrees (e.g., between about 55 and about 65 degrees, or between about 50 degrees and about 70 degrees) over a single stride. A competitive sprinter may have a thigh angle that various about 80 degrees (e.g., between about 75 and about 85 degrees, or between about 70 degrees and about 90 degrees) over a single stride. The thigh position as a function of percentage of a stride is typically similar for similar speeds as well. Based on the function of the thigh position as a function of stride, the angular velocity can be estimated (e.g., as a derivative or an approximation of the derivate of the function of the thigh position).

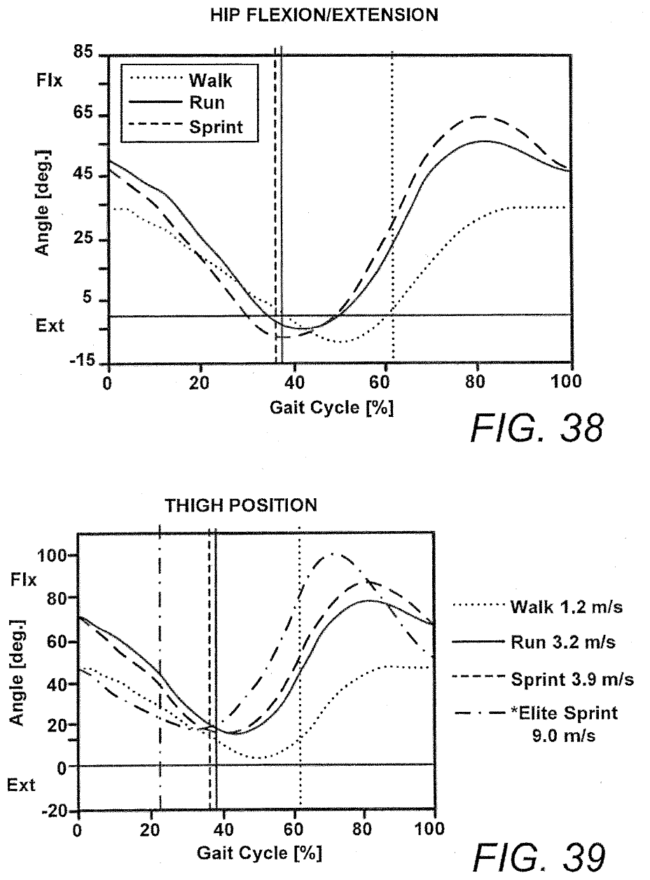

For example, FIGS. 38 and 39 illustrate typical behavior of a thigh during a stride or gait cycle as a function of percentage of the gait cycle. In each of the figures, each graph begins and ends at initial contact, representing one full gait cycle along the x-axis. Additionally, in each of the figures, walking is represented by the dotted line, running is represented by the solid line, and sprinting is represented by the dashed line. Similarly, the toe off point for each gait is represented by a vertical line of the same line style. FIG. 38 illustrates a graph of the hip flexion and extension where the angle represents the position of the femur relative to the position of the pelvis. FIG. 39 illustrates a graph of the position of the thigh relative to the vertical. For this graph, 0 degrees indicates that the thigh is in a vertical position. In FIG. 39, an additional gait is included, that of an elite sprinter. As can be seen from FIGS. 38 and 39, the typical thigh position of an adult varies smoothly and predictably for walkers (dotted line), runners (solid line), and sprinters (dashed line).

The resistance units can be configured to provide an indication of differences in average or instantaneous power. For example, the instantaneous power determined with the resistance unit can be provided as an indication of the difference in power being exerted relative to the power being exerted at a previous reference time. As another example, the instantaneous power determined with the resistance unit can be provided as an addition to an estimate of the total power exerted by a wearer while walking, running, or sprinting. Basic trend information such as increasing, decreasing or steady power output can be displayed to the athlete and/or the coach.

In various implementations, an estimate or determination of the total power or energy exerted by a wearer while walking, running, or sprinting can be provided by an equation that relates typical mechanical energy exerted by a person to running speed. The running (or walking) speed of the wearer can be estimated based on a stride rate and a stride length of the wearer. The stride length can be directly measured by measuring a distance run and measuring a number of strides taken over the distance. The stride length is then the distance divided by the number of strides. As another example, the stride length can be estimated based on average stride lengths of runners based on a runner's height. The stride length of a walking adult can be estimated as about 62 inches (where stride refers to two steps), or between about 52 and about 62 inches, between about 48 and about 66 inches, between about 45 and about 70 inches, or between about 44 and about 72 inches. The stride length of a walking adult can be estimated as the height of the person multiplied by 0.413-0.415. For sprinters, the stride length can be estimated as typically between about 1.14 times the person's height to about 1.35 times the person's height. The stride length of a running adult can be estimated to be between about 50 inches and about 100 inches, between about 55 inches and about 95 inches, between about 58 inches and about 93 inches, or between about 60 inches and about 90 inches. In some embodiments, the estimated stride length for a female can be different from an estimated stride length for a male. For example, for long distance runners, the average stride length for a female can be estimated to be between about 53 inches and about 63 inches and for a male it can be between about 72 inches and about 88 inches. Similarly, for sprinters, the average stride length for a female can be estimated to be between about 67 inches and about 81 inches and for a male it can be between about 83 inches and about 103 inches.

The typical total mechanical energy exerted by a person while running can be determined based on the speed of the runner, the weight of the runner, and/or the stride rate of the runner. In various implementations, the mechanical energy exerted by a person while running can be calculated based on a speed of the runner using a statistical relationship. An example statistical relationship of the work done by a person's body, W (in Joules), running at a speed, x (in meters per second), can be: W=440+170(x−3.3). The variation on this relationship can be between about 10% to about 15% (e.g., the actual mechanical energy has a 68% likelihood of being within 15% of the calculated value using the above relationship). Another example statistical relationship of the work done by a person's body normalized to the weight of the person, Wkg (in Joules/kg), can be: Wkg=7.5+3(x−3.3). The variation on this relationship can be between about 8% to about 12% (e.g., the actual mechanical energy has a 68% likelihood of being within 12% of the calculated value using the above relationship). Another example statistical relationship of the work done by a person's body normalized to the weight of the person and to their stride rate, Wtime (in Joules/kg/s), can be: Wtime=10.5+5.5(x−3.3). The variation on this relationship can be between about 7% to about 10% (e.g., the actual mechanical energy has a 68% likelihood of being within 10% of the calculated value using the above relationship).

In some embodiments, the mechanical energy can be used to determine estimated total power exerted while running. This value can be used as a baseline energy or power and the measurements provided by the resistance units can be used as an addition to this calculated energy or power to provide to the wearer an estimate of the energy or power exerted while walking, running, and/or sprinting. In certain embodiments, the measurements provided by the resistance units can be provided as a percentage of the total mechanical energy exerted by the wearer.

In general, a wide variety of information can be calculated on board and relayed to the wearer, to the wearer and a coach, or to the coach alone for display. Alternatively raw data or partially processed data may be exported to a wearer's remote device, and computations performed thereon. In either event, information such as actual step count, actual distance traveled for walking, near actual distance traveled for running, actual stride length, actual stride rate and real time ratios discussed below can be displayed to the wearer, in many instances more accurately than conventional activity trackers which must in many cases estimate metrics with more or less accuracy.

Certain ratios or relationships can be determined and displayed in real time, and/or saved for later study. For example, power to weight ratio, expressed as watts per kilogram can really be derived and displayed. The controller may be configured to generate for display the trend line over a time interval such as one week, one month, over the season or longer. An athlete can observe an improvement resulting from either a weight loss, an increase in power output, or probably most likely some of both.

Power to heart rate ratio may also be derived and displayed, and utilized for example to determine aerobic decoupling. Aerobic endurance is a critical factor in achieving success as an endurance athlete. Thus, it can be an important training tool to understand whether you have reached an optimal aerobic fitness level. When aerobic endurance improves, there is a reduced upward heart rate drift relative to a constant power output. The reverse is also true that when heart rate is held steady during extensive endurance training, output may be expected to drift downward. This relationship between heart rate and power output is referred to as coupling. The extent of decoupling can be quantitatively evaluated during workout in two different ways. If an endurance event is undertaken in such a manner that maintains a steady heart rate, the rate of downward power drift can be monitored. Alternatively, since incremental power (power drift) can be determined essentially in real time in accordance with the present invention, an athlete can focus on maintaining a steady power output and view what happens to heart rate over the measurement period. Excessive decoupling (too steep a heart rate climb at constant power output or too steep a power decline at constant heart rate) would indicate a lack of aerobic endurance fitness. The controller may be configured to generate comparative displays of most recent efficiency test with the same test on a prior occasion. The prior occasion may be at least one day, one week, one month, one season or one year or more (e.g., lifetime to date) previously. This information can be utilized to reinforce the value of or modify any of a variety of variables ranging from different types and intensities of training to diet, body weight among others.

An athlete can also utilize the present invention to determine an ideal (e.g., running or cycling) pace. If an athlete is exerting a constant power output, but heart rate is climbing, that exertion level may be acceptable for a short burst but is not sustainable over the long term. Thus the athlete should back down to a lower exertion level. Alternatively, if at a constant power heart rate is declining, the athlete knows that they have a reserve and can afford the energy expense of elevating their exertion level.

Another derived metric that can be determined by the controller for display is efficiency factor. Efficiency factor is normalized power divided by average heart rate over a set interval. By comparing efficiency factor data points over time, such as comparing a present value to a value determined at least one week ago, one month ago, from the beginning of the season, at least a year ago or other interval, one would hope to see an improvement in efficiency factor and can also observe the rate of improvement over time. One will see an improvement in efficiency factor either by experiencing a lower average heart rate for a given steady power output, or an increased power output for a given steady heart rate.

A block diagram showing functional components of an electronics unit 590 is shown in FIG. 33. Force sensor 600 is connected via wire or wireless interface 604. A sensor such as a Flexiforce sensor (obtained from Tekscan of South Boston, Mass., www.tekscan.com) may be used, having a conductance which is linear with force, and an analog interface 606 is used to generate an output voltage that is linear with the applied force. Other analog interfaces may not generate an output voltage that is linear with force, but they will generate a voltage that has a predetermined relationship to a force sensed by the force sensor. The analog interface 606 may contain a variable reference circuit for adjusting a range of the output voltage, depending on the desired performance. The voltage output by the analog interface 606 drives an analog-to-digital converter 608, which is controlled by a central processing unit (CPU) 610 and sampled at a known and constant rate. The CPU 610 may be, for example, a microprocessor or a digital signal processor. The CPU 610 is responsible for executing a power algorithm 612 that calculates the wearer's power exerted to overcome the resistance element based on force sensed by the force sensor 600. Data resulting from the calculation is transmitted to a remote electronics unit (activity tracker, cell phone, heads up display, wrist worn display, internet, etc.) by a radio frequency transmitter 614 and antenna 616 via a data channel. During calibration mode, calibration port 618 is used to interface to electronics unit 590. EEPROM memory 620 stores data generated during calibration. Operating power is supplied, for example, by a battery driven power supply, which is not shown but is very well known in the art. Some sensors are preferably calibrated (zeroed) and may be susceptible to drift with changing temperature. A temperature compensation circuit (not shown) is preferably included, to determine the temperature of the sensor and compensate for thermally induced error.

FIG. 34 is a block diagram showing functional components of a remote electronics unit that may display power or calories burned data to the wearer, coach or other application. An antenna 622 and a radio frequency receiver 624 receive data transmitted via the data channel. A CPU 626 controls the user interface, which may include a display 628 and potentially controls such as switches 630. Calibration data and user data are stored in EEPROM memory 632. During calibration mode, calibration port 634 is used to interface to the electronics unit. Operating power for the electronics unit may be supplied, for example, by a battery driven power supply, which is not shown but is very well known in the art. Additional details may be found in U.S. Pat. No. 7,599,806 to Hauschildt, the disclosure of which is hereby incorporated in its entirety herein by reference.

Power may be displayed as real time data, peak, average, rolling average or integrated over a predetermined interval of time (e.g., 10 second, 30 second, I minute or more). Display may be visual, such as on a smart phone, activity tracker or other hand held, wrist worn or mounted device. Power may alternatively be displayed on a heads up display such as an eyeglass with heads up display, or audibly over an audio output using a text to voice converter or tactiley via vibrator or electrical stimulation. Display may be configured to provide an indication of crossing a preset value such as when power output moves either above or below a preset upper or lower alarm limit to allow the wearer to control their power output to within a preset zone.

Referring to FIG. 35 there is illustrated a simplified bilateral system to implement the present invention indicated generally by the reference numeral 640. A left leg power module 642 and a right leg power module 644 are indicated by dotted lines and are in communication with a control and display unit 646, for example over a radio link 648 (e.g., ANT+, Bluetooth, Zigbee or others disclosed elsewhere herein). Each module 642, 644 comprises of one or more force sensor(s) 650, an accelerometer 652 and related measurement electronics 654 carried by each module. The display and control unit 646, usually battery powered, can be attached to any convenient place such as the wrist of the wearer, handlebar or other display as has been discussed. The connection between the sensors and electronics in the module and the sensors and electronics elsewhere on or in communication with the garment or wearer may be by wired conductors on or integrated into the garment, or may be by a wireless link such as radio protocols described elsewhere herein or by electromagnetic induction.

In a preferred embodiment the communication between the power module electronics embedded in the resistance module and the display and control unit is by a radio link 648. Each of left leg power module 642 and right leg power module 644 uses the radio to transmit a set of measurement data at one or more fixed points on each stride. In operation each of the power modules 642, 644 transmits its data in a short burst when the stride reaches a fixed point in its cycle, such as at the heel strike or toe roll off. Because the two strides are 180 degrees away from each other, data transmission can be timed to ensure that the transmissions from each power module assembly will never interfere with each other. Each burst of data contains a set of samples or measurements taken at regular intervals during the stride cycle, and may include force, proximity, cadence, femoral (or other) extension angle, heel strike, toe off, and accelerometer information. Each sample has an associated timestamp, which may be explicit or implicit, to specify its time relationship to the other samples in the set and to other sets of samples. The electronics in the power modules may include processing of the data before it is transmitted to the control unit 646. Additional details may be found in U.S. Pat. No. 8,762,077 to Redmond, et al., the disclosure of which is hereby incorporated in its entirety herein by reference.

It may be desirable to monitor the wearer's oxygen saturation, and/or CO2, to evaluate the transition between aerobic and anaerobic threshold as well as the effect on that threshold of varying the degree of resistance provided by the resistance unit (by adjusting an adjustable resistance unit or switching resistance units having different resistance levels). A sensor may be configured to be placed in contact with the wearer such as by permanent or removable attachment to the garment, or independent attachment to the wearer. The sensor may be configured to obtain a plethysmography signal, although it should be understood that any device configured to obtain oxygen saturation and/or heart rate data may be used in accordance with the techniques of the present disclosure. The system may include a monitor in communication with the sensor. The sensor and the monitor may communicate wirelessly as shown, or may communicate via one or more cables (e.g., the sensor and the monitor may be coupled via one or more cables). The sensor may include a sensor body, which may support one or more optical components, such as one or more emitters configured to emit light at certain wavelengths through a tissue of the subject and/or one or more detectors configured to detect the light after it is transmitted through the tissue of the subject.

The sensor may include one or more emitters and/or one or more detectors. The emitter may be configured to transmit light, and the detector may be configured to detect light transmitted from the emitter into a patient's tissue after the light has passed through the blood perfused tissue. The detector may generate a photoelectrical signal correlative to the amount of light detected. The emitter may be a light emitting diode, a superluminescent light emitting diode, a laser diode or a vertical cavity surface emitting laser (VCSEL). Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the emitter may be used to measure blood oxygen saturation, water fractions, hematocrit, or other physiological parameters of the patient. In certain embodiments, the emitter may emit at least two (e.g., red and infrared) wavelengths of light. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

The detector may be an array of detector elements that may be capable of detecting light at various intensities and wavelengths. In one embodiment, light enters the detector after passing through the tissue of the wearer. In another embodiment, light emitted from the emitter may be reflected by elements in the wearer's tissue to enter the detector. The detector may convert the received light at a given intensity, which may be directly related to the absorbance and/or reflectance of light in the tissue of the wearer, into an electrical signal. That is, when more light at a certain wavelength is absorbed, less light of that wavelength is typically received from the tissue by the detector, and when more light at a certain wavelength is transmitted, more light of that wavelength is typically received from the tissue by the detector. After converting the received light to an electrical signal, the detector may send the signal to the monitor, where physiological characteristics may be calculated based at least in part on the absorption and/or reflection of light by the tissue of the wearer.

As indicated above, the monitoring system may be configured to monitor the wearer's oxygen saturation and/or heart rate during exercise. The system may also be configured to determine whether the wearer is utilizing an aerobic or an anaerobic pathway based at least in part on the athlete's oxygen saturation and/or heart rate. For example, the monitoring system may compare the athlete's oxygen saturation and/or heart rate to one or more zones corresponding to various types of exercise (e.g., aerobic exercise and anaerobic exercise) to determine whether the wearer is utilizing the aerobic or the anaerobic pathways. Each of the one or more zones may be defined by a percentage or a range of percentages of oxygen saturation and/or a value or a range of values of heart rate, and each of the one or more zones may have an upper limit and a lower limit for oxygen saturation and/or heart rate. For example, a first zone may include an oxygen saturation range and/or a heart rate range corresponding to aerobic exercise, while a second zone may include an oxygen saturation range and/or heart rate range corresponding to anaerobic exercise. A visual, audio and/or tactile display or feedback may be provided to the wearer to indicate status and/or change in status between an aerobic metabolism level of activity and an anaerobic metabolism level of activity. Additional implementation details may be found in US patent publication No. 2015/0031970 to Lain, entitled Systems and Methods for Monitoring Oxygen Saturation During Exercise, the disclosure of which is hereby incorporated by reference in its entirety herein.

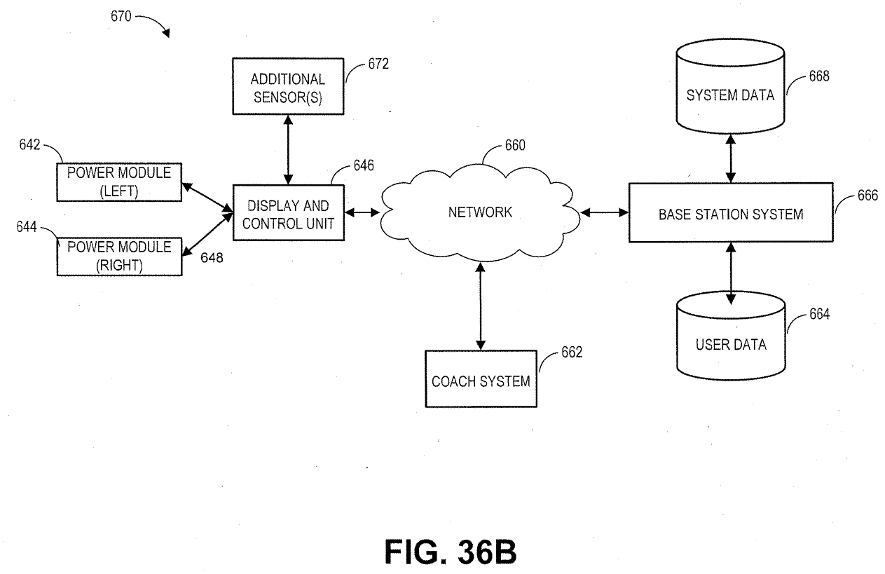

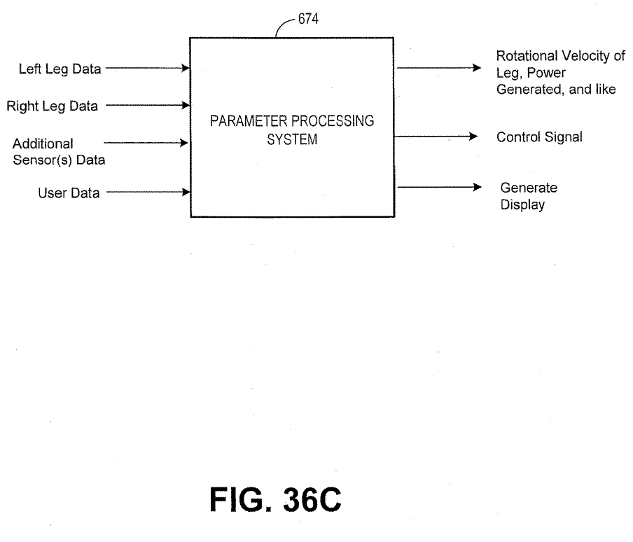

FIG. 36B illustrate an electronic computing environment 670 including interaction and communication between multiple electronic systems. As discussed above, the biometric units such as power modules 642 and 646 can transmit data, which is captured by the force sensor(s) 650 and other sensors herein, to a display control unit 646 over communications link 642 and 648. These communications links 642 and 648 can include radio links for wireless transmission of the captured data to the display and control unit 646. In some embodiments, the display and control unit 646 can include a mobile device including an antenna for receiving the transmitted captured data. In some embodiments, the display and control unit 646 can send instructions to the power modules 642 and 644 to transmit the captured data. The display and control unit 646 can also manage bandwidth and power requirements during transmission.

The display and control unit 646 can include a memory to store the received data from the power modules 642 and 644. The display and control unit 646 can further include one or more receiving and transmitting antennas and one or more hardware processors. In some embodiments, the display and control unit 646 can also receive data from addition sensor(s) 672. As discussed above, additional sensor(s) 672 can include an oxygen saturation detecting sensor, a heart rate detector, or the like. The display and control unit 646 can transmit the stored data over a network 660 to a base station system 666. The network 660 may be a local area network (LAN), a wide area network (WAN), cellular network, such as the Internet, combinations of the same, or the like. The transmission of data from the display and control unit 646 to the base station system 666 may be automatic or based on a user input.

The base station system 666 can include one or more servers for implementing and executing a parameter processing system 674 as discussed below. The base station system 666 can also include one or more data repositories 664 and 668. These data repositories can store user specific data, including historical data received from the display and control unit 646. The data repositories can also store predefined system parameters including threshold conditions and constants. The electronic computing environment 670 can also include a coach system 662 capable of receiving the captured data and output generated by the parameter processing system discussed below. The coach system 662 can include computing devices, including mobile electronic devices as discussed herein.

FIG. 36C illustrates an embodiment of a parameter processing system 674 for generating one or more electronic outputs based on received data. In some embodiments, the parameter processing system 674 controls operations of the power module 642 and 644. Operations can include capturing data, transmission of captured data, and other operations described herein. The parameter processing system 674 include programming instructions for the control and generation of output. Accordingly, the programming instructions correspond to the processes and functions described herein. The programming instructions can be stored in a memory of the base station system 666. In some embodiments, the programming instructions can also be stored in the display and control unit 646, coach system 662, such that some or all aspects of the parameter processing system 674 can be implemented in the display and control unit 646 and/or the coach system 662. The parameter processing system 674 can be executed by one or more hardware processors in the base station system 666, coach system 662, or display and control unit 646, or a combination. The programming instructions can be implemented in C, C++, JAVA, or any other suitable programming languages. In some embodiments, some or all of the portions of the parameter processing system 674 can be implemented in application specific circuitry such as ASICs and FPGAs.

FIG. 36C illustrates example inputs and outputs generated by the parameter processing system 674. In some embodiments, the parameter processing system 674 can electronically derive total power expended, peak power, power per stride, declining power reserve and other parameters discussed herein. The parameter processing system can also generate displays for display on a coach system 662 or the display and control unit 646. In some embodiments, the parameter processing system 662 generates output in near real time, with minimal delay from the time that the data was captured. In an embodiment, the delay is less than 1 second. The generated display can include power and heart rate ratio data. The parameter processing system 674 can accumulate historic data and determine an average to generate a baseline for the total power expended per play, per game, marathon, or physical event. The generated display can show a declining remaining power based on the stored historical performance. The display can include display elements, such as a gauge with scales based on the historical performance and the current meter based on captured data.

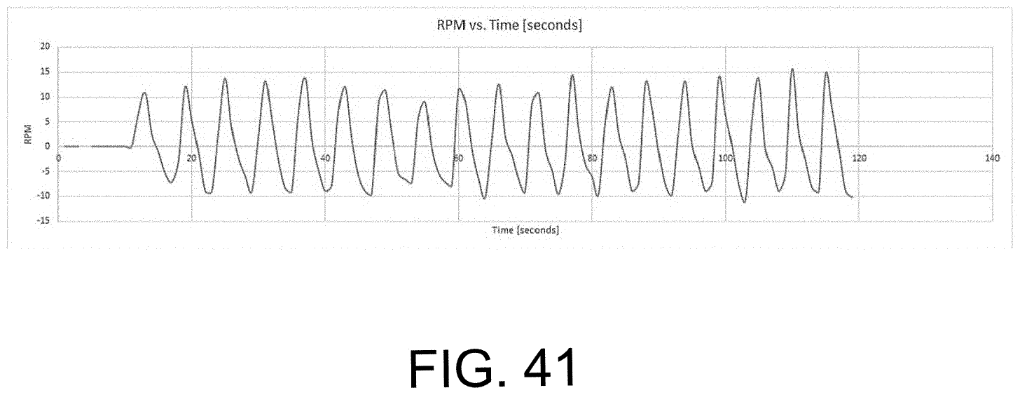

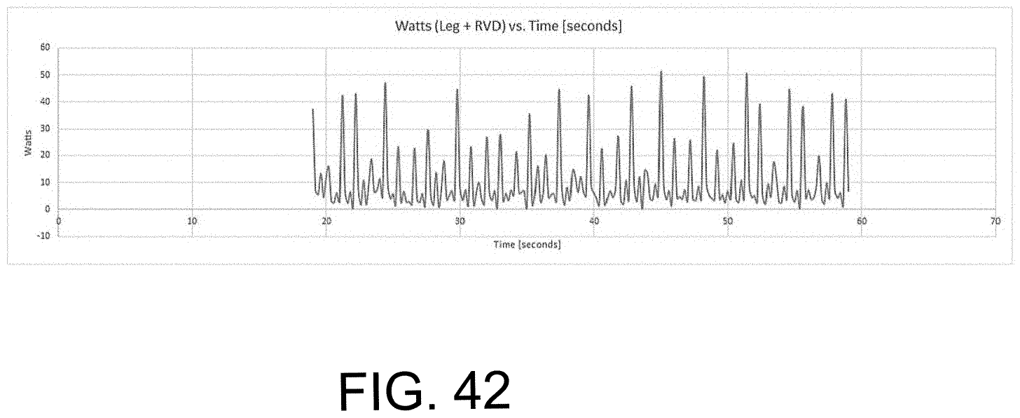

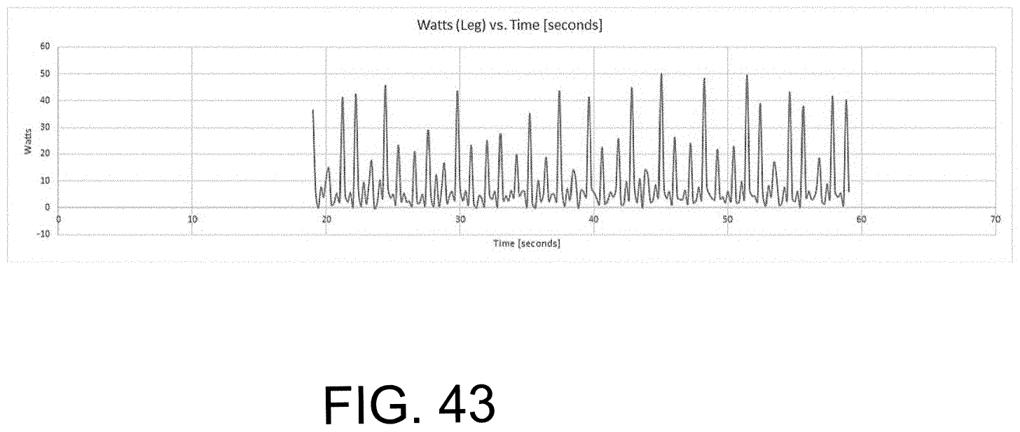

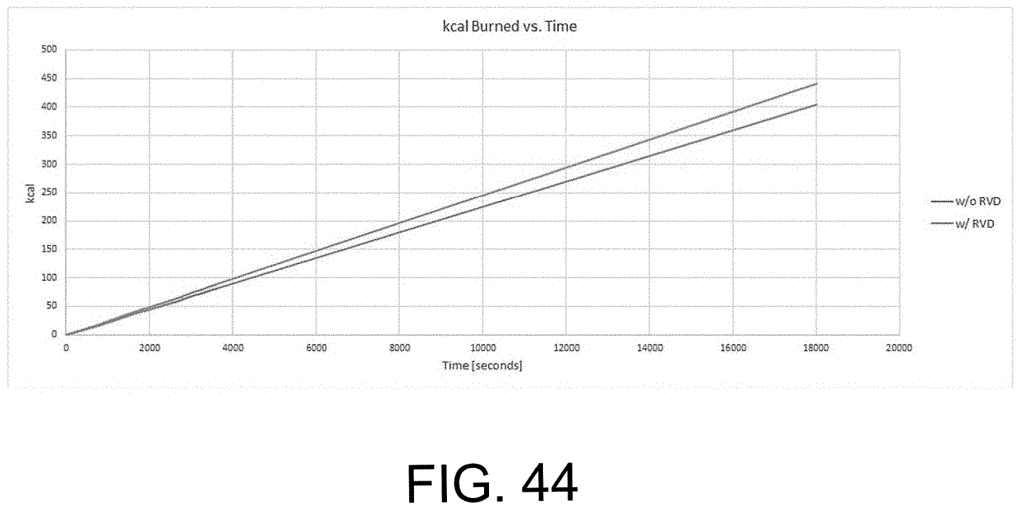

FIG. 40 describes a leg rotation with respect to time captured by a rotary encoder. FIG. 41 describes rotational velocity in RPM of a leg with respect to time in an embodiment. Rotational velocity in RPM may be calculated as an instantaneous slope at each point of the graph in FIG. 40. FIG. 42 is a graph depicting power generated by rotation of a leg with respect to time in an embodiment. Power generated by rotation of a leg may be calculated from mass, angular acceleration, and angular velocity of a leg. FIG. 43 is a graph depicting power generated by RVD type resistance units and rotation of a leg with respect to time in an embodiment. FIG. 44 is a graph depicting cumulative power generated by RVD type resistance units with respect to time in an embodiment. These graphs can be generated by the parameter processing system 674 for display.

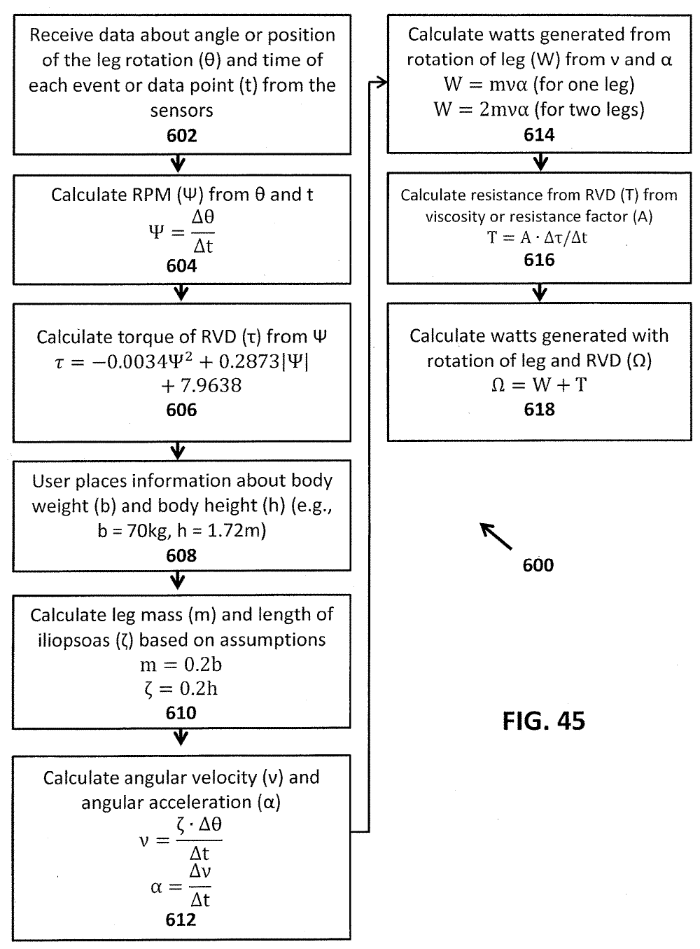

Referring to FIG. 45, the flow chart 600 describe steps to calculate rotational velocity of a leg, torque of RVD type resistance units, angular velocity, angular acceleration, power generated by rotation of a leg or two legs, and power generated from RVD type resistance units as performed in an embodiment. The flow chart 600 can be implemented by the parameter processing system 674 as programmed instructions. In step 602, the parameter processing system 674 receives data about angle or position of the leg rotation (θ) and time of each event or data point (t) from one or more sensors. Then, in step 604, the parameter processing system 674 calculates rotational velocity in RPM (Ψ) based on angle of the leg rotation (θ) and time (t) according to the following equation.

$$Ψ = \frac{Δθ}{Δ t}$$

In step 606, the parameter processing system 674 torque of RVD type resistance units (τ) based on rotational velocity in RPM (Ψ) according to the following equation.

$$\begin{matrix}τ = - 0.0034 {Ψ}^{2} + 0.2873 \vert Ψ \vert + 7.9638 & when Ψ \neq 0 \\ τ = 0 & when Ψ = 0\end{matrix}$$

In step 608, the user of the device places information about body weight (b) and body height (h). Alternatively, the device may set a default value for body weight (b) and body height (h) (e.g., b=70 kg; h=1.72 m for an average male). Then, the parameter processing system 674 calculates leg mass (m) and the length of iliopsoas (Q according to the following equations, as shown in step 610.

$$m = 0.2 b$$

$$ζ = 0. 2 k$$

These equations are based on assumptions on the relationships between body weight (b) and leg mass (m) and between body height (h) and the length of iliopsoas (ζ). A process may use different equations to calculate leg mass and the length of iliopsoas.

In step 612, the parameter processing system 674 calculates angular velocity (v) and angular acceleration (α) based on the following equations:

$$v = \frac{ζ \cdot Δθ}{Δ t}$$

$$α = \frac{Δ v}{Δ t}$$

Then, in step 614, the parameter processing system 674 calculates power generated from rotation of a leg or two legs (W) from angular velocity (v) and angular acceleration (α) according to the following equations.

$$W = mv α for one leg$$

$$W = 2 mv α for two legs$$

In step 616, the parameter processing system 674 calculates resistance from RVD type resistance units (T) from viscosity or resistance factor (A) according to the following equation.

$$T = A \cdot Δ τ / Δ t$$

Then, in step 618, the parameter processing system 674 calculates power generated from rotation of a leg or two legs and RVD type resistance units (0) according to the following equation.

$$Ω = W + T$$

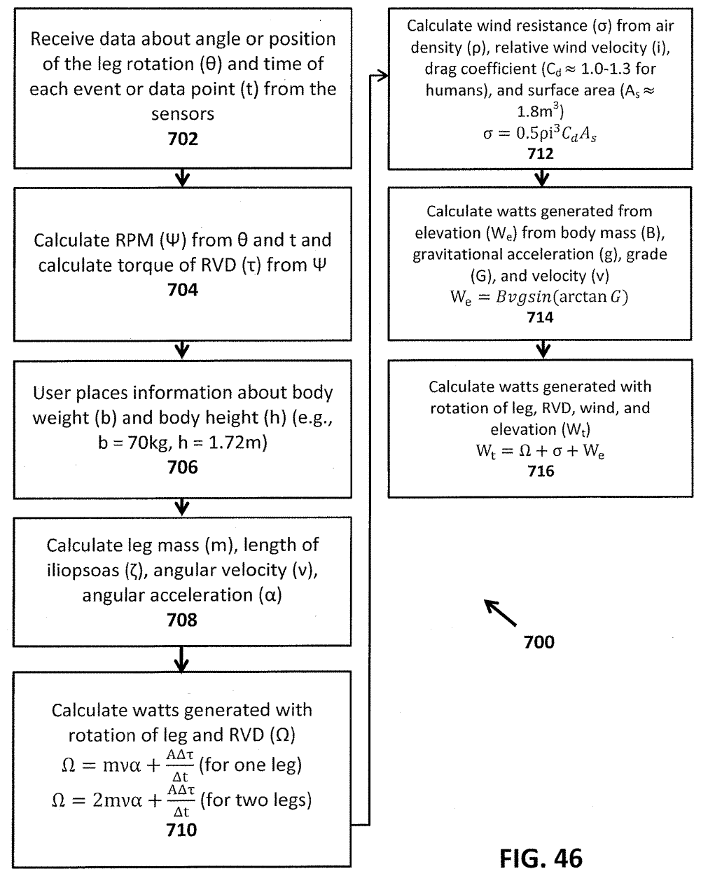

The the parameter processing system 674 may implement additional functionalities to calculate power adjusted with respect to elevation and/or wind. Referring to FIG. 46, the flow chart 700 describe steps to calculate rotational velocity of a leg, torque of RVD type resistance units, angular velocity, angular acceleration, power generated by rotation of a leg or two legs, power generated by RVD type resistance units, power generated from wind resistance, and power generated from elevation as performed in an embodiment. The flow chart 700 can be implemented by the parameter processing system 674 as programmed instructions.

In step 702, the parameter processing system 674 of the device receives data about angle or position of the leg rotation (θ) and time of each event or data point (t) from one or more sensors. Then, in step 704, the parameter processing system 674 calculates rotational velocity in RPM (T) based on angle of the leg rotation (θ) and time (t) (e.g., according to the equation used in step 604) and calculates torque of RVD type resistance units (T) based on rotational velocity in RPM (T) (e.g., according to the equation used in step 606).

In step 706, the parameter processing system 674 can receive user input corresponding to body weight (b) and body height (h). Alternatively, the parameter processing system 674 may set a default value for body weight (b) and body height (h) (e.g.,b=70 kg; h=1.72 m for an average male). Then, in step 708, the parameter processing system 674 calculates leg mass (m) and the length of iliopsoas (ζ) (e.g., according to the equations used in step 610) and calculates angular velocity (v) and angular acceleration (a) (e.g., according to the equations used in step 612).

In step 710, the parameter processing system 674 calculates power (0) generated from rotation of a leg or two legs (W) and RVD type resistance units (T) from angular velocity (v), angular acceleration (α), and viscosity or resistance factor (A) according to the following equations.

$$Ω = W + T = mv α + \frac{A Δ τ}{Δ t} ( for one leg )$$

$$Ω = W + T = 2 mv α + \frac{A Δ τ}{Δ t} ( for two legs )$$

Then, in step 712, the parameter processing system 674 calculates wind resistance (σ) from air density (φ, relative wind velocity (i), drag coefficient (C_{d}≈1.0-1.3 for humans), and surface area (Aₛ≈1.8 m³ for humans) according to the following equation.

$$σ = 0.5 ρ {i}^{3} {C}_{d} {A}_{s}$$

In step 714, the parameter processing system 674 calculates power generated from elevation (Wₑ) from body mass (B), gravitational acceleration (g), grade (G), and velocity (v) according to the following equation. Grade (G) is defined as rise over run, or a change in vertical distance over a change in horizontal distance during elevation.

$${W}_{e} = Bvgsin ( arc tan G )$$

In step 716, the parameter processing system 674 calculates power generated from rotation of a leg or two legs, RVD type resistance units, wind resistance, and elevation according to the following equation.

$${W}_{t} = Ω + σ + {W}_{e} = W + T + σ + {W}_{e}$$

The indicators may be configured to allow the user to access data recorded and/or calculated by the device. The data may comprise short interval (e.g., 1, 3, 5, 7, or 10 seconds) average power, peak power, total cumulative watts expended, stride length, stride rate, cumulative distance travelled (i.e., sum of the strides), and power-to-heart-rate ratio. The display and control unit 646 or the coach system 662 may further include one or more screens configured to display data or information to the user. The screens may comprise a LCD screen, an OLED screen, a touch screen, or any other screen known in the art. In another aspect, the device may further comprise one or more indicators to communicate data or information to the user. The indicators may include a visible LED, an audible buzzer, a display screen, or any other user interface known in the art. The device may include a heart rate monitor configured to measure the heart rate of the user or import heart rate data from a separate heart rate monitor. The parameter processing system 674 may calculate power-to-heart-rate ratio based on the power calculated by the parameter processing system 674 and the heart rate.

The parameter processing system 674 can generate (for capture in memory and/or display) different metrics of stride depending upon the intended activity of a wearer group. For example, running or walking endurance athletes may benefit from tracking instantaneous values, cumulative values or previous selected activity values for any one or more of pace, stride count, stride cadence (steps per minute or rpm), stride length, total power, horizontal power, vertical power (and/or vertical oscillation; both reflecting running efficiency), instantaneous power, peak power (different durations), and actual distance traveled. Instantaneous power may be the average power recorded over a preceding time interval such as for example within the range of from about 5 seconds to one minute, or 15 seconds to 30 seconds. Ground contact time (heel strike to toe off) may also be helpful in determining running efficiency (generally lower is better because it may reflect a more efficient use of energy). An indicium of the onset of fatigue may be displayed or otherwise expressed to the runner, when ground time starts to climb relative to air time in excess of a preset threshold value. All of the foregoing may be displayed as a trend over time, to enable the athlete or coach to evaluate fitness, fatigue, sustainable pace, and inefficiency due to deterioration in form.

Certain ratios may also be generated, such as Watts/Kg of body weight; heart rate vs. power ratio; speed/watt; and metrics of sustainable output such as functional threshold power. This reflects the average power output at a relatively constant exertion level for a unit of time. A runner might run for an hour at a sustainable pace and the average power expended may be 300 Watts. That runner may then train sets at 110% or 120% or more of the 300 W threshold. During a run, the parameter processing system 674 may signal the runner (visual display; audible sound; tactile vibrator) if the power exerted drifts above or below preset triggers. For example a trigger might be set at any time the power deviates from a target by at least about 2% or 5% or 7% or 10% or 25% or 20% or more. The target may be the functional threshold power, or any target set by the runner or derived by the parameter processing system 674 from historical data. For example, the parameter processing system 674 may maintain cumulative average power data from prior marathons. For the next marathon, that cumulative average value becomes the target value so the runner is given a signal whenever power output deviates from within the desired range from the historical power value.

The parameter processing system 674 may also be configured to capture hip angle and power data such as from a rotary encoder type sensor. This can be used to determine the hip angle at which speed per watt is the greatest which can lead to running posture changes to improve efficiency.

Any of the foregoing can be expressed as combined left leg and right leg data, and deviations can be identified and signaled or displayed to reveal bilateral asymmetries which may suggest corrective training. Any or all of the foregoing can be downloaded to a training program configured to provide post event analytics.

Devices configured for team sports (e.g., football, soccer, ice hockey, basketball, etc.) might be configured to capture metrics like stride count, distance travelled (based upon stride count stride length and inseam) peak power, peak accelerations and total power. Data may be captured and transmitted to a coaching software for diagnostic evaluation.

Although disclosed primarily in the context of lower body garments, any of the resistance elements and attachment fabrics and structures disclosed herein can be adopted for use for any other motion segment on the body, including the shoulder, elbow, wrist, neck, abdomen (core) and various other motion segments of the upper body. Any of the various resistance elements and attachment structures disclosed herein can be interchanged with any other, depending upon the desired performance. In addition, the present invention has been primarily disclosed as coupled to a type of garment resembling a complete article of clothing. However any of the resistance systems disclosed herein may be carried by any of a variety of other types of garments including braces, wearable clothing subassemblies, straps, cuffs or other wearable support construct that is sufficient to mechanically couple one or more resistance or data capturing elements to the body and that may be worn over or under or integrated into conventional clothing.

1. A wearable garment training system for monitoring stride biomechanics at the hip, comprising:
a waist portion;
a left leg portion;
a right leg portion;
a left hip biomechanics unit removably carried by a left connector on the garment and aligned with a rotational axis of the left leg;
a right hip biomechanics unit removably carried by a right connector on the garment and aligned with a rotational axis of the right leg;
wherein the left and right biomechanics units each capture data for enabling the determination of power expended by a wearer throughout a range of motion at the hip.

2. A training system as in claim 1, wherein at least one of the biomechanics units is configured to capture time and angle data during flexion.

3. A training system as in claim 1, wherein at least one of the biomechanics units is configured to capture time and angle data during extension.

4. A training system as in claim 1, wherein at least one of the biomechanics units is configured to measure force applied during flexion.

5. A training system as in claim 1, wherein at least one of the biomechanics units is configured to measure force applied during extension.

6. A training system as in claim 1, wherein at least one of the biomechanics units is configured to capture data relating to angular velocity of a wearer's leg throughout the range of motion.

7. A training system as in claim 1, further comprising a processor, for determining power expended throughout the range of motion.

8. A training system as in claim 1, further comprising a transmitter, for transmitting data to a remote device.

9. A training system as in claim 2, further comprising a transmitter, for transmitting time and angle data to a remote device.

10. A training system as in claim 1, further comprising a left knee biomechanics unit and a right knee biomechanics unit.

11. A training system as in claim 1, wherein the left and right hip biomechanics units each further comprise rotatable resistance units.

12. A training system as in claim 1, wherein each biomechanics unit comprises a housing and a femoral lever extending from the housing.

13. A training system as in claim 1, wherein the garment comprises a compression fabric.

14. A training system as in claim 13, wherein the fabric comprises a polyester elastane fabric with moisture wicking properties.

15. A training system as in claim 11, wherein the left and right resistance units each impose a resistance of at least about 5 inch pounds.

16. A training system as in claim 15, wherein the left and right resistance units each impose a resistance of at least about 10 inch pounds.

17. A training system as in claim 16, wherein the left and right resistance units each impose a resistance of at least about 15 inch pounds.

18. A training system as in claim 1, wherein the garment comprises a wearable harness.

19. A training system as in claim 18, wherein the harness comprises a waist band and left and right leg bands.

20. A training system as in claim 1, further comprising an ANT+ transmitter.

21.-42. (canceled)
